(19) United States
(12) Patent Application Publication
(10) Pub. No.: US 2014/0142714 A1
(43) Pub. Date: May 22, 2014

(54) KNEE PROSTHESIS ASSEMBLY HAVING PROPORTIONAL CORONAL GEOMETRY

(71) Applicants: Abraham P. Wright, Winona Lake, IN (US); Joseph G. Wyss, Fort Wayne, IN (US); Christel M. Wagner, Plymouth, IN (US); Mark A. Heldreth, Mentone, IN (US); Danny W. Rumple, Jr., Warsaw, IN (US)

(72) Inventors: Abraham P. Wright, Winona Lake, IN (US); Joseph G. Wyss, Fort Wayne, IN (US); Christel M. Wagner, Plymouth, IN (US); Mark A. Heldreth, Mentone, IN (US); Danny W. Rumple, Jr., Warsaw, IN (US)

(21) Appl. No.: 13/683,630

(22) Filed: Nov. 21, 2012

(51) Int. Cl. A61F 2/38

(52) U.S. Cl. CPC A61F 2/3836
USPC 623/20.31

(57) ABSTRACT

An orthopaedic knee prosthesis assembly includes a plurality of femoral components. Each component includes a medial condyle and a lateral condyle. When each component is viewed in a coronal plane extending through a distal-most point of the medial condyle and a distal-most point of the lateral condyle, the medial condyle has a medial curved distal-most surface that includes the distal-most point, and a width is defined between the distal-most points of the medial condyle and the lateral condyle. The coronal radius of the distal-most surface of a first component is proportionally greater than the coronal radius of a second component. The coronal radius of the second component is proportionally greater than the coronal radius of a third component. The first component width is proportionally greater than the second component width, and the second component is proportionally greater than the width of the third component.

### CROSS-REFERENCE TO RELATED APPLICATIONS

Cross-reference is made to U.S. patent application Ser. No. ______ entitled “Knee Prosthesis Assembly Having Proportional Trochlear Groove Geometry” by Abraham P. Wright et al., which was filed on Nov. 21, 2012 and is expressly incorporated herein by reference.

### TECHNICAL FIELD

The present disclosure relates generally to orthopaedic prostheses, and particularly to orthopaedic prostheses for use in knee replacement surgery.

### BACKGROUND

Joint arthroplasty is a well-known surgical procedure by which a diseased and/or damaged natural joint is replaced by a prosthetic joint. One type of knee prosthesis includes a tibial tray, a femoral component, and a polymer insert or bearing positioned between the tibial tray and the femoral component. Depending on the severity of the damage to the patient's joint, orthopaedic prostheses of varying mobility may be used. For example, the knee prosthesis may include a “fixed” tibial bearing in cases wherein it is desirable to limit the movement of the knee prosthesis, such as when significant soft tissue damage or loss is present. Alternatively, the knee prosthesis may include a “mobile” tibial bearing in cases wherein a greater degree of freedom of movement is desired. Additionally, the knee prosthesis may be a total knee prosthesis designed to replace the femoral-tibial interface of both condyles of the patient's femur or a uni-compartmental (or uni-condylar) knee prosthesis designed to replace the femoral-tibial interface of a single condyle of the patient's femur.

The knee prosthesis may also include a patella component that is secured to the patient's natural patella such that its posterior surface articulates with the femoral component during extension and flexion of the knee. Types of patella components include a dome-shaped polymer bearing and a conforming or anatomic bearing that is designed to conform with the bearing surfaces of the femoral component.

The type of orthopedic knee prosthesis used to replace a patient's natural knee may also depend on whether the patient's posterior cruciate ligament is retained or sacrificed (i.e., removed) during surgery. For example, if the patient's posterior cruciate ligament is damaged, diseased, and/or otherwise removed during surgery, a posterior stabilized knee prosthesis may be used to provide additional support and/or control at later degrees of flexion. Alternatively, if the posterior cruciate ligament is intact, a cruciate retaining knee prosthesis may be used.

### SUMMARY

According to one aspect of the disclosure, an orthopaedic knee prosthesis assembly is disclosed. The orthopaedic knee prosthesis assembly includes a plurality of femoral components, and each component includes a medial condyle and a lateral condyle. When each component is viewed in a coronal plane extending through a distal-most point of the medial condyle and a distal-most point of the lateral condyle, the medial condyle has a medial distal-most surface that is curved and includes the distal-most point of the medial condyle, and a medial inner surface connected to the medial distal-most surface and extending proximally away from the medial distal-most surface. The medial distal-most surface has a coronal radius of curvature. When each component is viewed in a coronal plane extending through a distal-most point of the medial condyle and a distal-most point of the lateral condyle, the lateral condyle has a lateral distal-most surface that includes the distal-most point of the lateral condyle, and a lateral inner surface connected to the lateral distal-most surface and extending proximally away from the lateral distal-most surface. An angle is defined between the medial inner surface and the lateral inner surface. The plurality of femoral components include a first component, a second component, and a third component, and the angles of the first, second, and third components are equal in magnitude. The coronal radius of the first component is greater than the coronal radius of the second component by a scale factor, and the coronal radius of the second component is greater than the coronal radius of the third component by the scale factor.

In some embodiments, the scale factor may be equal to approximately 1.041. In some embodiments, when each component is viewed in the coronal plane, the lateral distal-most surface may be curved and may have a coronal radius of curvature that is equal to the coronal radius of curvature of the medial distal-most surface.

In some embodiments, the scale factor may be a first scale factor. When each component is viewed in the coronal plane, a width may be defined between the distal-most point of the medial condyle and the distal-most point of the lateral condyle. The width of the first component may be greater than the width of the second component by a second scale factor different from the first scale factor, and the width of the second component may be greater than the width of the third component by the second scale factor. In some embodiments, the second scale factor is equal to approximately 1.024.

In some embodiments, the magnitude of each of the angles of the first, second, and third components may be approximately 130 degrees. Additionally, in some embodiments, when each component is viewed in the coronal plane, the medial condyle may have a medial rounded edge surface that is connected to the medial inner surface and extend proximally away from the medial inner surface, the lateral condyle may have a lateral rounded edge surface that is connected to the lateral inner surface and extend proximally away from the lateral inner surface, and an arced imaginary line may extend between the medial condyle and the lateral condyle and have a radius of curvature. The arced imaginary line may define a first tangent point at the transition between the medial rounded edge surface and the medial inner surface and a second tangent point at the transition between the lateral rounded edge surface and the lateral inner surface. The radii of curvature of the arced imaginary lines of the first, second, and third components may be equal.

In some embodiments, the radius of curvature of the arced imaginary line of each of the first, second, and third components may be equal to approximately 14 millimeters. In some embodiments, when each component is viewed in the coronal plane, the medial condyle may have a medial flat surface that is connected to the medial rounded edge surface and extend proximally away from the medial rounded edge surface, the lateral condyle have a lateral flat surface that is connected to the lateral rounded edge surface and extend proximally away from the lateral rounded edge surface, and each component may include an intercondylar notch defined between the medial flat surface and the lateral flat surface.

Additionally, in some embodiments, when each femoral component is viewed in the coronal plane, the arced imaginary line, the medial inner surface, and the lateral inner surface may define a trochlear groove of the component. The trochlear groove has a depth, and the depth of the trochlear groove of the first component may be greater than the depth of the trochlear groove of the second component. The depth of the trochlear groove of the second component may be greater than the depth of the trochlear groove of the third component.

In some embodiments, when each femoral component is viewed in the coronal plane, the arced imaginary line has an apex, and the depth of the trochlear groove may be defined between the distal-most point of the medial condyle and the apex of the arced imaginary line.

According to another aspect, an orthopaedic knee prosthesis assembly includes a plurality of femoral components, and each component includes a medial condyle and a lateral condyle. When each component is viewed in a coronal plane extending through a distal-most point of the medial condyle and a distal-most point of the lateral condyle, the medial condyle has a medial distal-most surface that is curved and includes the distal-most point of the medial condyle, and a medial inner surface extending proximally away from the medial distal-most surface. The medial distal-most surface has a coronal radius of curvature. When each component is viewed in a coronal plane extending through a distal-most point of the medial condyle and a distal-most point of the lateral condyle, the lateral condyle has a lateral distal-most surface that includes the distal-most point of the lateral condyle, and a lateral inner surface extending proximally away from the lateral distal-most surface. An arced imaginary line has a first tangent point on the medial inner surface and a second tangent point on the lateral inner surface, a first imaginary line extends through the first tangent point of the arced imaginary line and a third tangent point that is defined at a transition between the medial inner surface and the medial distal-most surface, and a second imaginary line extends through the second tangent point of the arced imaginary line and a fourth tangent point that is defined at a transition between the lateral inner surface and the lateral distal-most surface. An angle is defined between the first imaginary line and the second imaginary line.

The plurality of femoral components includes a first component, a second component, and a third component. The angles of the components are equal in magnitude, the coronal radius of the first component is greater than the coronal radius of the second component by a scale factor, and the coronal radius of the second component is greater than the coronal radius of the third component by the scale factor.

In some embodiments, the scale factor may be equal to approximately 1.041. Additionally, in some embodiments, when each component is viewed in the coronal plane, a width may be defined between the distal-most point of the medial condyle and the distal-most point of the lateral condyle. The width of the first component may be greater than the width of the second component by a second scale factor, and the width of the second component may be greater than the width of the third component by the second scale factor.

In some embodiments, each arced imaginary line may have a radius of curvature, and the radii of curvature of the arced imaginary lines of the first, second, and third components may be equal. In some embodiments, the magnitude of each of the angles of the plurality of femoral components may be approximately 130 degrees, and the radii of curvature of each of the arced imaginary lines of the plurality of femoral components may be approximately 14 millimeters.

According to another aspect, an orthopaedic knee prosthesis assembly includes a plurality of femoral components, and each component includes a medial condyle and a lateral condyle. When each component is viewed in a coronal plane extending through a distal-most point of the medial condyle and a distal-most point of the lateral condyle, the medial condyle has a medial curved distal-most surface that includes the distal-most point, and the medial curved distal-most surface has a coronal radius of curvature. A width is defined between the distal-most points of the medial condyle and the lateral condyle. The plurality of femoral components include a first, second, and third component, and the coronal radius of the first component is greater than the coronal radius of the second component by a first scale factor. The coronal radius of the second component is greater than the coronal radius of the third component by the first scale factor. The width of the first component is greater than the width of the second component by a second scale factor that is less than the first scale factor, and the width of the second component is greater than the width of the third component by the second scale factor.

In some embodiments, the first scale factor may be equal to 1.041. Additionally, in some embodiments, the second scale factor may be equal to 1.024. In some embodiments, when each femoral component is viewed in the coronal plane, the medial condyle may have a medial inner surface extending proximally away from the medial curved distal-most surface and a medial rounded edge surface extending proximally away from the medial inner surface. An arced imaginary line may have a first tangent point at a transition between the medial rounded edge surface and the medial inner surface. The arced imaginary line may have a radius of curvature. The radii of curvature of the arced imaginary lines of the first, second, and third components may be equal.

According to one aspect, an implantable orthopaedic knee prosthesis assembly is disclosed. The implantable orthopaedic knee prosthesis assembly includes a femoral component including an articular surface configured to engage a tibial bearing and a trochlear groove defined in the articular surface. The trochlear groove is angled laterally when the femoral component is viewed in an anterior elevation view. The implantable orthopaedic knee prosthesis assembly also includes a patella component received in the trochlear groove, and the patella component is positioned at a first location in the trochlear groove at a first degree of flexion, and a second location in the trochlear groove at a second degree of flexion. The second degree of flexion is greater than the first degree of flexion and in a range of about 0 degrees to about 30 degrees. An arced imaginary line defines a central section of the trochlear groove. When the femoral component is viewed in a first coronal plane extending through the first location, the arced imaginary line has a first radius of curvature, and when the femoral component is viewed in a second coronal plane extending through the second location, the arced imaginary line has a second radius of curvature that is less than the first radius of curvature.

In some embodiments, the second radius of curvature may be greater than 15.5 millimeters. Additionally, in some embodiments, the first radius of curvature may be equal to approximately 27 millimeters.

In some embodiments, the femoral component may include a patellar surface that defines the trochlear groove. The patellar surface may extend between a medial edge connected to the articular surface and a lateral edge connected to the articular surface. When the femoral component is viewed in the first coronal plane, a first imaginary line may extend through a point on the medial edge and may be tangent to the arced imaginary line, a second imaginary line may extend through a point on the lateral edge and is tangent to the arced imaginary line, and a first angle may be defined between the first imaginary line and the second imaginary line. When the femoral component is viewed in the second coronal plane, a third imaginary line may extend through a point on the medial edge and is tangent to the arced imaginary line, a fourth imaginary line may extend through a point on the lateral edge and may be tangent to the arced imaginary line, and a second angle may be defined between the third imaginary line and the fourth imaginary line. The second angle may have a magnitude less than the first angle.

In some embodiments, the magnitude of the second angle may be greater than or equal to 132 degrees. Additionally, in some embodiments, the first angle may have a magnitude equal to approximately 152 degrees.

In some embodiments, the patella component may be positioned at a third location in the trochlear groove at a third degree of flexion that is greater than or equal to 45 degrees. When the femoral component is viewed in a third coronal plane extending through the third location, the arced imaginary line defining the central section of the trochlear groove may have a third radius of curvature that is less than the second radius of curvature.

In some embodiments, the patella component may be positioned at a fourth location in the trochlear groove at a fourth degree of flexion that is greater than the third degree of flexion and less than 90 degrees. When the femoral component is viewed in a fourth coronal plane extending through the fourth location, the arced imaginary line may have a fourth radius of curvature that is equal to the third radius of curvature. In some embodiments, the third radius may be equal to approximately 14 millimeters.

Additionally, in some embodiments, when the femoral component is viewed in the fourth coronal plane, a fifth imaginary line may extend through a point on the medial edge and is tangent to the arced imaginary line, a sixth imaginary line may extend through a point on the lateral edge and may tangent to the arced imaginary line, and a third angle may be defined between the fifth imaginary line and the sixth imaginary line. The third angle may have a magnitude less than the second angle. In some embodiments, the third angle may have a magnitude equal to approximately 130 degrees.

In some embodiments, when the femoral component is viewed in a sagittal plane, a second arced imaginary line may define the central section of the trochlear groove. The second arced imaginary line may have a constant radius of curvature.

According to another aspect, an implantable orthopaedic knee prosthesis assembly includes a plurality of femoral components. Each femoral component includes an articular surface configured to engage a tibial bearing, a trochlear groove defined in the articular surface, the trochlear groove having a longitudinal axis, and a pair of medial and lateral condyles. When each femoral component is viewed in a coronal plane extending through a distal-most point of the medial condyle and a distal-most point of the lateral condyle, the medial condyle includes a medial inner surface that partially defines the trochlear groove, the lateral condyle includes a lateral inner surface that partially defines the trochlear groove, a sulcus angle is defined between the medial inner surface and the lateral inner surface, and a width is defined between the distal-most point of the medial condyle and the distal-most point of the lateral condyle. When each femoral component is viewed in an anterior elevation view, the distal-most point of the medial condyle and the distal-most point of the lateral condyle are positioned in a distal plane, an imaginary axis extends orthogonal to the distal plane, and a trochlear angle is defined between the longitudinal axis and the imaginary axis. The sulcus angles of the each of the plurality of femoral components are equal in magnitude, the width of each femoral component is different from the width of each of the other femoral components, and the magnitudes of the trochlear angles vary inversely with the widths of the femoral components.

In some embodiments, the implantable orthopaedic knee prosthesis assembly may further include a patella component received in the trochlear groove of at least one of the femoral components. The patella component may be positioned at a first location in the trochlear groove of the femoral component at a first degree of flexion, and a second location in the trochlear groove of the femoral component at a second degree of flexion. The second degree of flexion may be greater than the first degree of flexion and in a range of about 0 degrees to about 30 degrees. A curved surface may define a central section of the trochlear groove at the first degree of flexion and the second degree of flexion. When the femoral component is viewed in a first coronal plane extending through the first location, the curved surface may have a first radius of curvature, and when the femoral component is viewed in a second coronal plane extending through the second location, the curved surface may have a second radius of curvature that is less than the first radius of curvature.

In some embodiments, the first radius may be equal to approximately 27 millimeters. The second radius may be equal to approximately 15.5 millimeters.

Additionally, in some embodiments, When each femoral component is viewed in the coronal plane extending through the distal-most point of the medial condyle and the distal-most point of the lateral condyle, the medial condyle may have a medial distal-most surface that includes the distal-most point of the medial condyle, and the medial distal-most surface may be curved and may have a coronal radius of curvature. The coronal radius of curvature each of the femoral components may increase proportionally with the width of each of the femoral components.

According to another aspect, an implantable orthopaedic knee prosthesis is disclosed. The implantable orthopaedic knee prosthesis includes a femoral component including an articular surface configured to engage a tibial bearing and a laterally-angled trochlear groove defined in the articular surface. The trochlear groove of the femoral component is configured to receive a patella component in a first location at a first degree of flexion and a second location at a second degree of flexion that is greater than the first degree of flexion and in a range of about 0 degrees to about 30 degrees. An arced imaginary line defines a central section of the trochlear groove. When the femoral component is viewed in a first coronal plane extending through the first location, the arced imaginary line has a first radius of curvature, and when the femoral component is viewed in a second coronal plane extending through the second location. The arced imaginary line has a second radius of curvature that is less than the first radius of curvature.

In some embodiments, the trochlear groove may be defined between a medial edge and a lateral edge. When the femoral component is viewed in the first coronal plane, a first imaginary line may extend through a point on the medial edge and may be tangent to the arced imaginary line. A second imaginary line may extend through a point on the lateral edge and is tangent to the arced imaginary line, and a first angle may be defined between the first imaginary line and the second imaginary line. When the femoral component is viewed in the second coronal plane, a third imaginary line may extend through a point on the medial edge and may be tangent to the arced imaginary line, a fourth imaginary line may extend through a point on the lateral edge and may be tangent to the arced imaginary line, and a second angle may be defined between the third imaginary line and the fourth imaginary line. The second angle may have a magnitude less than the first angle.

In some embodiments, the trochlear groove may be configured to receive a patella component in a third location at a third degree of flexion that is greater than or equal to 45 degrees. When the femoral component is viewed in a third coronal plane extending through the third location, the arced imaginary line defining the central section of the trochlear groove may have a third radius of curvature that is less than the second radius of curvature.

In some embodiments, when the femoral component is viewed in a sagittal plane, a second arced imaginary line may define the central section of the trochlear groove. The second arced imaginary line may have a constant radius of curvature.

### BRIEF DESCRIPTION OF THE DRAWINGS

The detailed description particularly refers to the following figures, in which:

FIG. 1 is a perspective view of an orthopaedic knee prosthesis assembly;

FIG. 2 is an anterior elevation view of the femoral component of FIG. 1;

FIG. 3 is an elevation view of a femoral component and a patella component of the orthopaedic knee prosthesis assembly of FIG. 1 showing the femoral component and the patella component articulated to one degree of flexion;

FIG. 4 is a coronal cross-sectional view of the femoral component of FIG. 3 taken along the line 4-4 in FIG. 3;

FIG. 5 is a coronal cross-sectional view similar to FIG. 4 showing the femoral component engaged with the patella component;

FIG. 6 is an elevation view similar to FIG. 3 showing the femoral component and the patella component articulated to another degree of flexion;

FIG. 7 is a coronal cross-sectional view of the femoral component taken along the line 7-7 in FIG. 6;

FIG. 8 is a coronal cross-sectional view similar to FIG. 7 showing the femoral component engaged with the patella component;

FIG. 9 is an elevation view similar to FIG. 3 showing the femoral component and the patella component articulated to another degree of flexion;

FIG. 10 is a coronal cross-sectional view of the femoral component taken along the line 10-10 in FIG. 9;

FIG. 11 is a coronal cross-sectional view similar to FIG. 10 showing the femoral component engaged with the patella component;

FIG. 12 is an elevation view similar to FIG. 3 showing the femoral component and the patella component articulated to another degree of flexion;

FIG. 13 is a coronal cross-sectional view of the femoral component taken along the line 13-13 in FIG. 12;

FIG. 14 is a cross-sectional view similar to FIG. 13 showing the femoral component engaged with the patella component;

FIG. 15 is an anterior elevation view showing the femoral component of FIGS. 1-14 and another larger femoral component;

FIG. 16 is a coronal cross-sectional view of the larger femoral component of FIG. 15;

FIG. 17 is a diagrammatic posterior elevation view of a number of differently-sized femoral components;

FIG. 18 is a table of one embodiment of dimensions of a family of femoral component sizes;

FIG. 19 is an elevation view of the femoral component of FIG. 1;

FIG. 20 is an elevation view of the tibial bearing of FIG. 1;

FIG. 21 is a graph of the anterior-posterior translation of the femoral component of FIG. 1;

FIGS. 22A-22J illustrate a table of one embodiment of radii of curvature values and sagittal conformity values for a family of femoral components and tibial bearings; and

FIGS. 23A-23J illustrate a table of another embodiment of radii of curvature and sagittal conformity values for a family of femoral components and tibial bearing.

### DETAILED DESCRIPTION OF THE DRAWINGS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

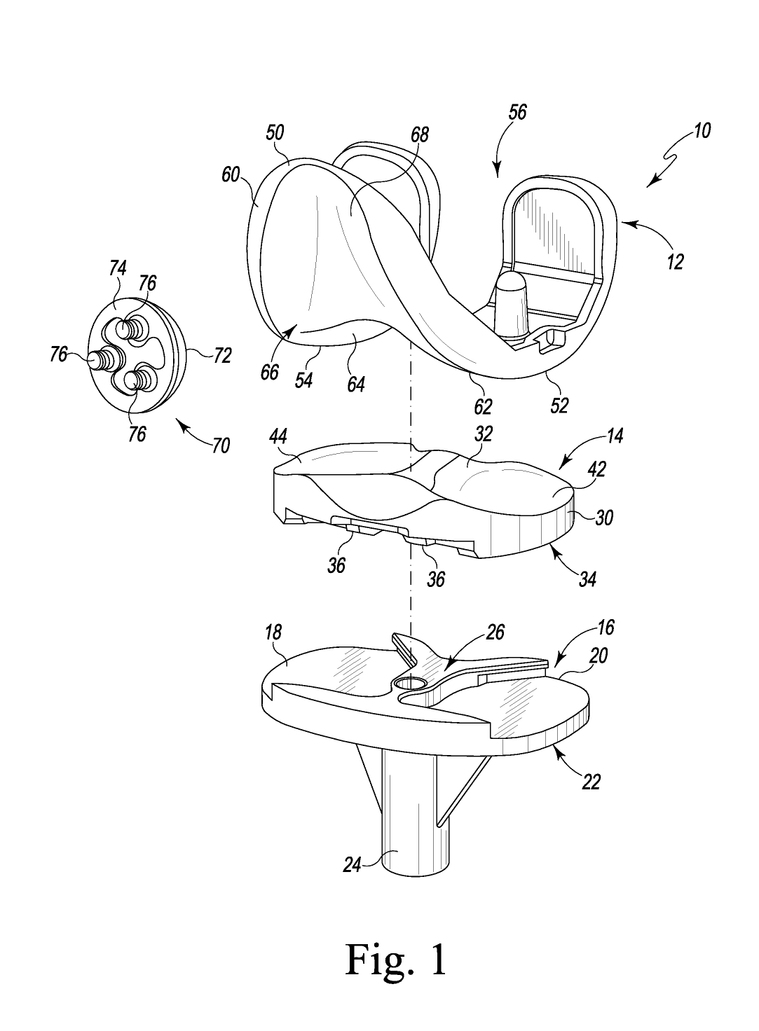

Referring now to FIG. 1, an orthopaedic knee prosthesis 10 is illustrated. The prosthesis 10 includes a femoral component 12, a tibial bearing 14, and a tibial tray 16. The femoral component 12 and the tibial tray 16 are illustratively formed from a metallic material such as cobalt-chromium or titanium, but may be formed from other materials, such as a ceramic material, a polymer material, a bio-engineered material, or the like, in other embodiments. The tibial bearing 14 is illustratively formed from a polymer material such as a ultra-high molecular weight polyethylene (UHMWPE), but may be formed from other materials, such as a ceramic material, a metallic material, a bio-engineered material, or the like, in other embodiments.

As described in more detail below, the femoral component 12 is configured to articulate with the tibial bearing 14, which is configured to be coupled with the tibial tray 16. As illustrated in FIG. 1, the tibial bearing 14 is embodied as a fixed tibial bearing, which is limited or restricted from rotating relative the tibial tray 16 during use. Examples of fixed bearing knee prostheses are described in U.S. Patent App. Pub. No. 2010/0063594 entitled “Fixed-Bearing Knee Prosthesis Having Interchangeable Components” by Stephen A. Hazebrouck et al., which was filed on Nov. 17, 2009, U.S. Patent App. Pub. No. 2009/0088859 entitled “Fixed-Bearing Knee Prosthesis Having Interchangeable Components” by Stephen A. Hazebrouck et al., which was filed on Sep. 28, 2007, U.S. Patent App. Pub. No. 2009/0082873 entitled “Fixed-Bearing Knee Prosthesis” by Stephen A. Hazebrouck et al., which was filed on Sep. 25, 2007 and is expressly incorporated herein by reference, U.S. patent application Ser. No. 13/247,453 entitled “Fixed Bearing Knee Prosthesis Having a Locking Mechanism with a Concave to Convex Mating Interface” by Stephen A. Hazebrouck et al., which was filed on Sep. 28, 2011, U.S. Patent App. Pub. No. 2011/0106268 entitled “Prosthesis for Cemented Fixation and Method for Making the Prosthesis” by Daren L. Deffenbaugh et al., which was filed on Oct. 14, 2010, and U.S. Patent App. Pub. No. 2011/0035018 entitled “Prosthesis with Composite Component” by Daren L. Deffenbaugh et al., which was filed on Oct. 14, 2010, each of which is expressly incorporated herein by reference. In other embodiments, the tibial bearing 14 may be embodied as a rotating or mobile tibial bearing that is configured to rotate relative to the tibial tray 16 during use. An example of a rotating platform knee prosthesis is described in U.S. Patent App. Pub. No. 2010/0016978 entitled “Antero-Posterior Placement of Axis of Rotation for a Rotating Platform” by John L. Williams et al., which was filed on Jul. 16, 2008 and is expressly incorporated herein by reference.

The tibial tray 16 is configured to be secured to a surgically-prepared proximal end of a patient's tibia (not illustrated). The tibial tray 16 may be secured to the patient's tibia via use of bone cement or other attachment means. The tibial tray 16 includes a platform 18 having a top surface 20 and a bottom surface 22. Illustratively, the top surface 20 is generally planar and, in some embodiments, may be highly polished. The tibial tray 16 also includes a stem 24 extending downwardly from the bottom surface 22 of the platform 18. A locking buttress 26 extends upwardly from the top surface 20. The buttress 26 is sized and shaped to receive a number of complimentary locking tabs of the tibial bearing 14, as described in greater detail below. An example of a tibial tray is described in U.S. Patent App. Pub. No. 2012/0109325 entitled “Tibial Component Having an Angled Cement Pocket” by Christel M. Wagner et al., which was filed on Sep. 30, 2011 and is expressly incorporated herein by reference.

As described above, the tibial bearing 14 is configured to be coupled with the tibial tray 16. The tibial bearing 14 includes a platform 30 having an upper bearing surface 32 and a bottom surface 34. As illustrated in FIG. 1, the bearing 14 includes a number of locking tabs 36 that extend from the platform 30. When the tibial bearing 14 is coupled to the tibial tray 16, the locking tabs 36 engage the buttress 26 of the tibial tray 16, thereby fixing the tibial bearing 14 to the tibial tray 16. In use, the tibial bearing 14 is fixed and not permitted to rotate relative to the tibial tray 16. In other embodiments, when the tibial bearing 14 is embodied as, for example, a mobile tibial bearing, the bearing 14 may include a stem that is received in a complimentary bore formed on the tibial tray 16. In such embodiments, the bearing is permitted to rotate about an axis relative to the tibial tray.

The upper bearing surface 32 of the tibial bearing 14 includes a medial bearing surface 42 and a lateral bearing surface 44. The bearing surfaces 42, 44 are configured to receive or otherwise contact corresponding medial and lateral condyles of the femoral component 12, as described in greater detail below. As such, each of the bearing surface 42, 44 has a concave contour that is shaped to receive one of the condyles of the femoral component 12.

The femoral component 12 is configured to be coupled to a surgically-prepared surface of the distal end of a patient's femur (not illustrated). The femoral component 12 may be secured to the patient's femur via use of bone cement or other attachment means. The femoral component 12 includes an anterior flange 50, a medial condyle 52, and a lateral condyle 54. The condyles 52, 54 are spaced apart to define an intercondylar notch 56 therebetween. An example of a femoral component is described in U.S. Patent App. Pub. No. 2012/0083894 entitled “Femoral Component of a Knee Prosthesis Having an Angled Cement Pocket” by Christel M. Wagner et al., which was filed on Sep. 30, 2010 and is expressly incorporated herein by reference.

The illustrative orthopaedic knee prosthesis 10 of FIG. 1 is embodied as a posterior cruciate-retaining knee prosthesis. That is, the femoral component 12 is embodied as a posterior cruciate-retaining femoral component 12 and the tibial bearing 14 is embodied as a posterior cruciate-retaining tibial bearing 14. It should be appreciated that in other embodiments the orthopaedic knee prosthesis 10 may be a posterior cruciate-sacrificing knee prosthesis. Examples of a posterior cruciate-retaining knee posterior knee prosthesis and a cruciate-sacrificing knee prosthesis are described in U.S. Patent App. Pub. No. 2009/0326667, entitled “Orthopaedic Femoral Component Having Controlled Condylar Curvature” by John L. Williams et al., which was filed on Jun. 30, 2008 and is hereby incorporated by reference.

Other examples of orthopaedic knee prostheses are described in U.S. Patent App. Pub. No. 2011/0178605 entitled “Knee Prosthesis System” by Daniel D. Auger et al., which was filed on Jan. 21, 2010, U.S. Patent App. Pub. No. 2011/0178606 entitled “Tibial Components for a Knee Prosthesis System” by Daren L. Deffenbaugh et al., which was filed on Jan. 21, 2010, U.S. Patent App. Pub. No. 2011/0029090 entitled “Prosthesis with Modular Extensions” by Anthony D. Zannis et al., which was filed on Oct. 14, 2010, U.S. Patent App. Pub. No. 2011/0035017 entitled “Prosthesis with Cut-off Pegs and Surgical Method” by Daren L. Deffenbaugh et al., which was filed on Oct. 14, 2010, U.S. Patent App. Pub. No. 2010/0036500 entitled “Orthopaedic Knee Prosthesis Having Controlled Condylar Curvature” by Mark A. Heldreth et al., which was filed on Jun. 19, 2009, U.S. Patent App. Pub. No. 2010/0016979 entitled “Knee Prosthesis With Enhanced Kinematics” by Joseph G. Wyss et al., which was filed on Jul. 16, 2008, U.S. Patent App. Pub. No. 2009/0326666 entitled “Posterior Stabilized Orthopaedic Knee Prosthesis” by Joseph G. Wyss et al., which was filed on Jun. 30, 2008, U.S. Patent App. Pub. No. 2009/0326665 entitled “Posterior Stabilized Orthopaedic Knee Prosthesis Having Control Condylar Curvature” by Joseph G. Wyss et al., which was filed on Jun. 30, 2008, U.S. Patent App. Pub. No. 2009/0326664 entitled “Posterior Cructiate Retaining Orthopaedic Knee Prosthesis Having Control Condylar Curvature” by Joseph G. Wyss et al., which was filed on Jun. 30, 2008, U.S. patent application Ser. No. 13/534,469 entitled “Posterior Stabilized Orthopaedic Knee Prosthesis Having Control Condylar Curvature” by Joseph G. Wyss et al., which was filed on Jun. 27, 2012, U.S. patent application Ser. No. 13/481,943 entitled “Positioning of Femoral Cam and Tibial Bearing Post to Reduce Anterior Sliding” by Joseph G. Wyss et al., which was filed on May 28, 2012, U.S. patent application Ser. No. 13/527,758 entitled “Posterior Stabilized Orthopaedic Prosthesis Assembly” by Joseph G. Wyss et al., which was filed on Jun. 20, 2012, U.S. patent application Ser. No. 13/534,459 entitled “Posterior Stabilized Orthopaedic Knee Prosthesis Having Control Condylar Curvature” by Joseph G. Wyss et al., which was filed on Jun. 27, 2012, U.S. patent application Ser. No. 13/487,990 entitled “Posterior Stabilized Orthopaedic Knee Prosthesis Having Control Condylar Curvature” by Christel M. Wagner et al., which was filed on Jun. 4, 2012, U.S. patent application Ser. No. 13/540,177 entitled “Orthopaedic Knee Prosthesis Having Controlled Condylar Curvature” by Christel M. Wagner et al., which was filed on Jul. 2, 2012, U.S. Patent App. Pub. No. 2008/0088860 entitled “Hinged Orthopaedic Prosthesis” by Alan Ritchie et al., which was filed on Sep. 30, 2007, and U.S. Patent App. Pub. No. 2008/0004708 entitled “Hinged Orthopaedic Prosthesis” by Joseph G. Wyss et al., which was filed on Jun. 30, 2006, each of which is expressly incorporated herein by reference. Cross-reference is also made to U.S. patent application Ser. No. 13/470,415 entitled “Prosthesis Kit with Finned Sleeve” by John Bonitati, which was filed on May 14, 2012 and is expressly incorporated herein by reference.

As illustrated in FIG. 1, the femoral component 12 has an articular surface 60 configured to engage the bearing surfaces 42, 44 of the tibial bearing 14. The articular surface 60 includes a medial condyle surface 62 of the medial condyle 52 and a lateral condyle surface 64 of the lateral condyle 54. The condyle surfaces 62, 64 are shaped to emulate the configuration of the patent's natural femoral condyles, and, as such, the surfaces 62, 64 are configured (e.g., curved) in a manner that mimics the condyles of the natural femur. In use, the condyle surfaces 62, 64 of the femoral component 12 articulate on the corresponding bearing surfaces 42, 44, respectively, of the tibial bearing 14 during extension and flexion of the patient's knee.

The femoral component 12 also includes a trochlear groove 66 that is defined in the articular surface 60. The trochlear groove 66 is configured to receive the patient's patella and is defined by a patellar surface 68, as described in greater detail below. In the illustrative embodiment, the prosthesis 10 includes a patella component 70 that is configured to be received in the trochlear groove 66 and articulate with the femoral component 12 during extension and flexion of the patient's knee. The patella component 70 is embodied as a monolithic polymer body constructed with a material that allows for smooth articulation between the patella component 70 and the femoral component 12. One such polymeric material is polyethylene such as ultrahigh molecular weight polyethylene (UHMWPE). It should be appreciated that in other embodiments the patella component 70 may be omitted from the prosthesis 10 such that the patient's natural patella is received in the trochlear groove 66 and articulates with the femoral component 12 during use.

As illustrated in FIG. 1, the patella component 70 includes a posterior bearing surface 72 that is configured to engage the patellar surface 68 of the femoral component 12. The patella component 70 also includes a flat anterior surface 74 having a number of fixation members, such as pegs 76, extending away therefrom. The pegs 76 are configured to be implanted into a surgically prepared posterior surface of the patient's natural patella (not illustrated). In such a way, the posterior bearing surface 72 of the patella component 70 faces toward the femoral component 12, thereby allowing the posterior bearing surface 72 to articulate with the patellar surface 68 during flexion and extension of the patient's knee.

As illustrated in FIG. 1, the patella component 70 is a dome patella component. As such, the posterior bearing surface 72 is dome-shaped. It should be appreciated that in other embodiments the patella component 70 may be an anatomic patella component. Examples of dome patella components and anatomic patella components are described in U.S. Patent App. Pub. No. 2012/0172994, entitled “Knee Prosthesis Having Cross-Compatible Dome and Anatomic Patella Components” by Abraham P. Wright et al., which is hereby incorporated by reference. Other examples of patella components are described in U.S. Patent App. Pub. No. 2012/0172993 entitled “Knee Prosthesis Having Commonly-Sized Patella Components With Varying Thicknesses” by Abraham P. Wright et al., which was filed on Dec. 30, 2010, U.S. Patent App. Pub. No. 2012/0123550 entitled “Implantable Patella Component Having a Thickened Superior Edge” by Abraham P. Wright et al., which was filed on Dec. 21, 2011, U.S. Patent App. Pub. No. 2009/0326662 entitled “Implantable Patella Component Having Thickened Superior Edge” by Abraham P. Wright et al., which was filed on Jun. 30, 2008, and U.S. Patent App. Pub. No. 2009/0326661 entitled “Implantable Patella Component Having Thickened Superior Edge” by Abraham P. Wright et al., which was filed on Jun. 30, 2008, each of which are expressly incorporated herein by reference.

It should be appreciated that the illustrative orthopaedic knee prosthesis 10 is configured to replace a patient's right knee; as such, the bearing surface 42 and the condyle 52 are referred to as being medially located, and the bearing surface 44 and the condyle 54 are referred to as being laterally located. However, in other embodiments, the orthopaedic knee prosthesis 10 may be configured to replace a patient's left knee. In such embodiments, it should be appreciated that the bearing surface 42 and the condyle 52 may be laterally located and the bearing surface 44 and the condyle 54 may be medially located. Regardless, the features and concepts described herein may be incorporated in an orthopaedic knee prosthesis configured to replace either knee joint of a patient.

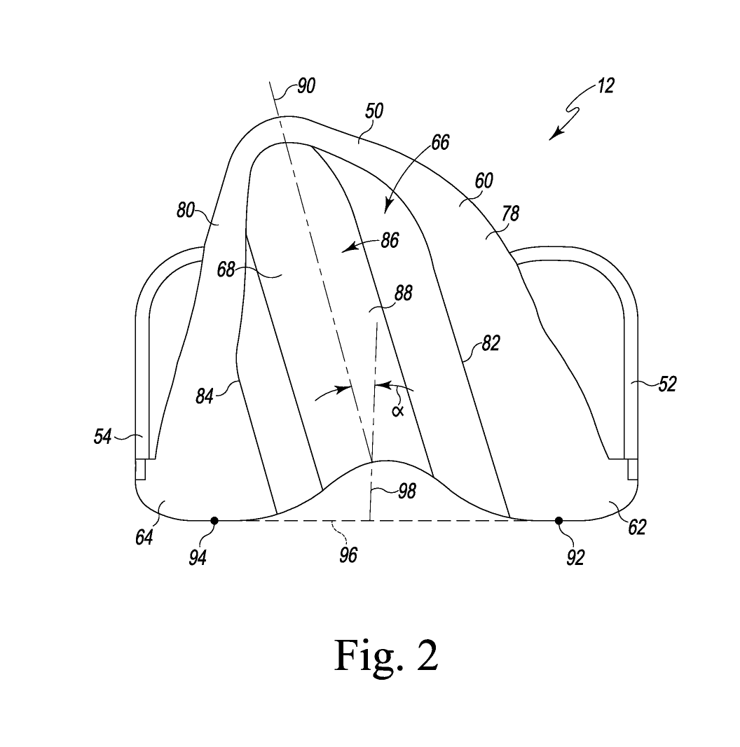

As described above, the femoral component 12 includes an articular surface 60. Referring now to FIG. 2, the articular surface 60 includes a medial anterior surface 78 and a lateral anterior surface 80 of the anterior flange 50. The medial anterior surface 78 transitions to the medial condyle surface 62 of the medial condyle 52, and the lateral anterior surface 80 of the lateral condyle 54 transitions to the lateral condyle surface 64 of the lateral condyle 54. The patellar surface 68 has a medial edge 82 that is connected to the medial anterior surface 78 and the medial condyle surface 62. The patellar surface 68 also has a lateral edge 84 that is connected to the lateral anterior surface 80 and the lateral condyle surface 64.

The trochlear groove 66 is defined in the articular surface 60 by the patellar surface 68 between the edges 82, 84 thereof. The trochlear groove 66 also includes a central section 86 defined by a bowed surface 88 of the patellar surface 68. As described in greater detail below, the trochlear groove 66 is angled laterally and includes a longitudinal axis 90 that extends laterally along the central section 86.

The medial condyle 52 has a distal-most point 92 on the medial condyle surface 62. Similarly, the lateral condyle 54 has a distal-most point 94 on the lateral condyle surface 64. As shown in FIG. 2, the distal-most points 92, 94 are positioned in a distal transverse plane 96, and an imaginary line 98 extends orthogonal to the plane 96. When the femoral component 12 is implanted, the imaginary line 98 extends parallel to the patient's inferior-superior axis (not shown).

A trochlear angle α of the trochlear groove 66 is defined between the longitudinal axis 90 and the imaginary line 98. In the illustrative embodiment, the trochlear angle α of the femoral component 12 has a magnitude of approximately 12.0 degrees. As such, the longitudinal axis 90 (and hence the trochlear groove 66) is angled laterally. It should be appreciated that in other embodiments the trochlear angle α may have a magnitude in the range of 10.1 degrees to 14.1 degrees, depending on, for example, the size of the femoral component.

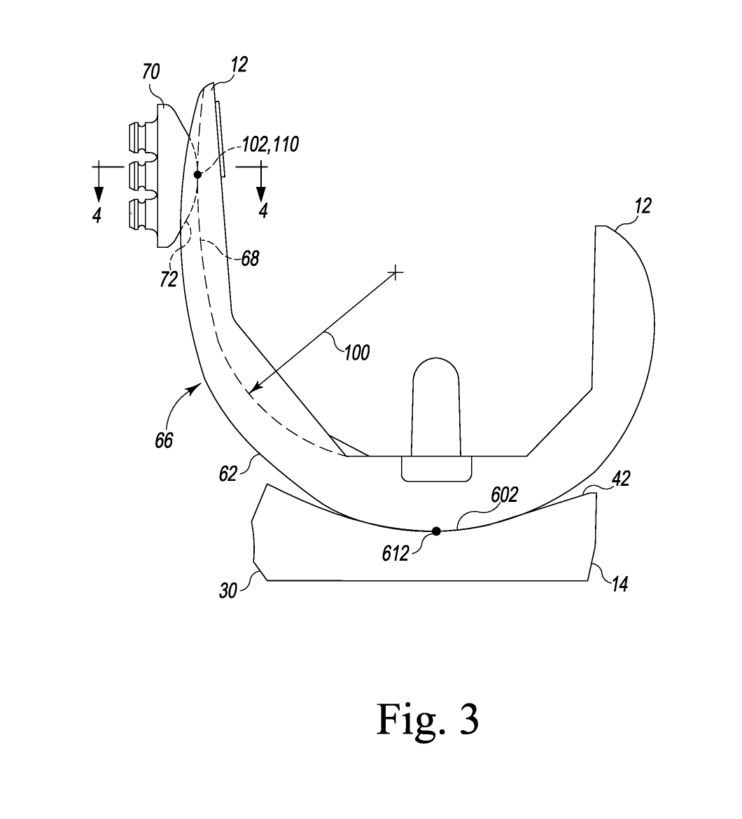

As shown in FIG. 3, the patellar surface 68 of the femoral component is convexly curved in the sagittal plane and is configured to contact the posterior bearing surface 72 of the patella component 70. The patellar surface 68 has a single radius of curvature 100. In the illustrative embodiment, the radius of curvature 100 is equal to approximately 35 millimeters. It should be appreciated that in other embodiments the radius of curvature 100 may be in the range of 24 millimeters to 43 millimeters.

Referring now to FIGS. 3-14, the patella component 70 is configured to be positioned in the trochlear groove 66. During flexion and extension of the patient's knee, the patella component 70 moves along the trochlear groove 66 and articulates on patellar surface 68 of the femoral component 12. For example, as illustrated in FIG. 3, when the orthopaedic knee prosthesis 10 is in extension or is otherwise not in flexion (e.g., a flexion of about 0 degrees), the patella component 70 is positioned in an anterior end of the trochlear groove 66 at a location 102.

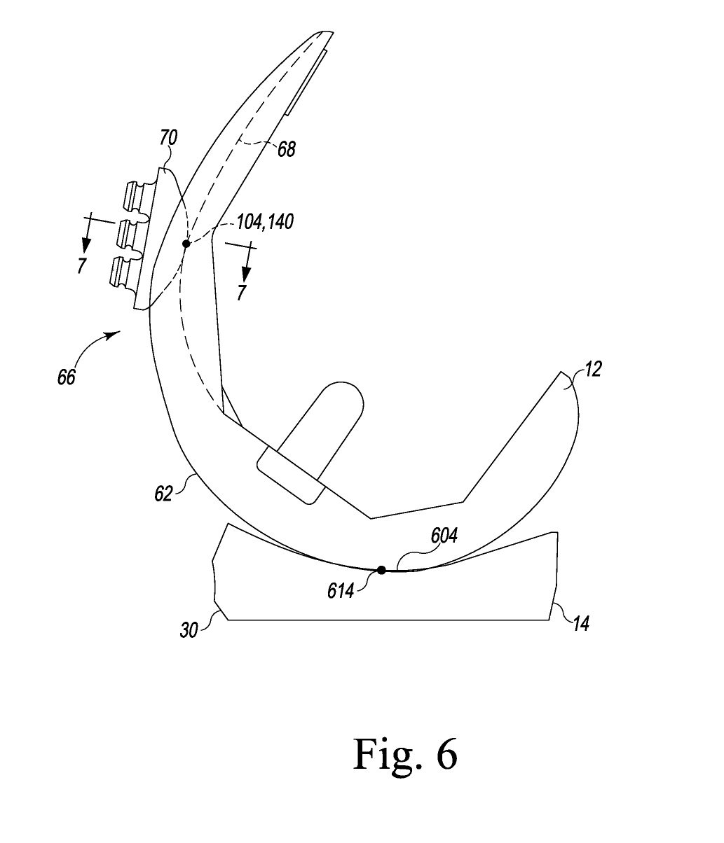

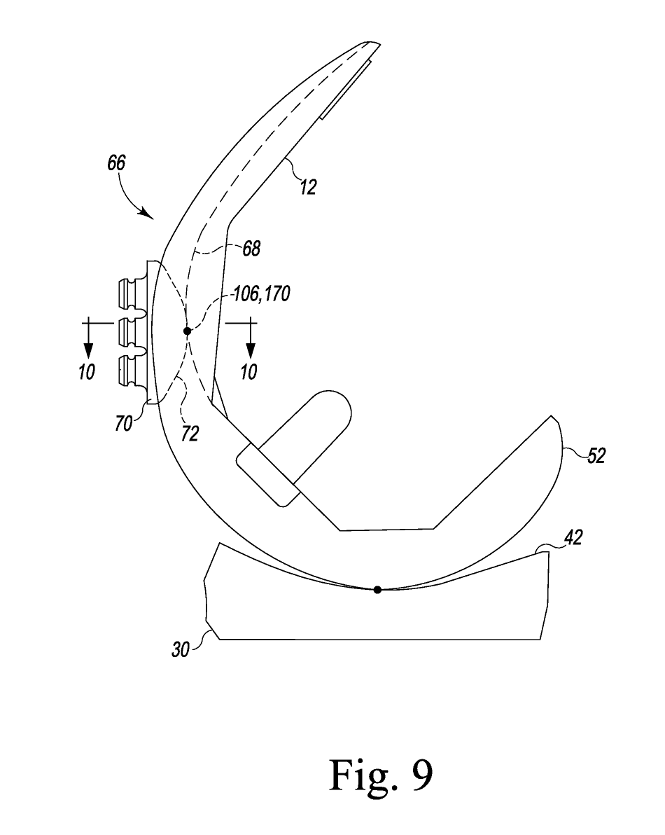

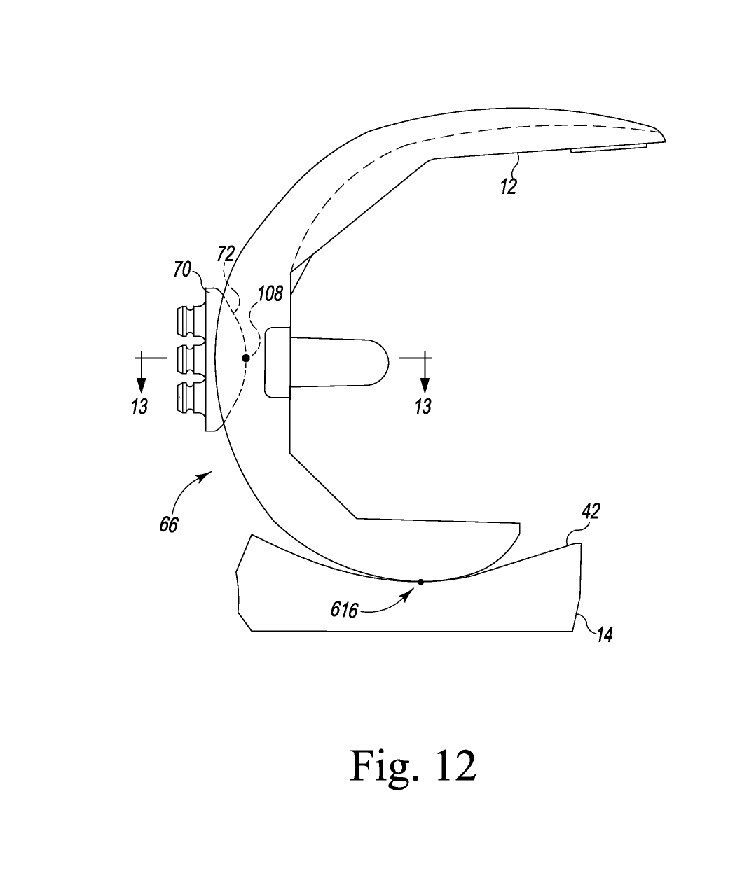

Additionally, as the orthopaedic knee prosthesis 10 is articulated through the middle degrees of flexion, the patella component 70 moves along the femoral component 12 to other locations in the trochlear groove 66. For example, as illustrated in FIG. 6, when the orthopaedic knee prosthesis 10 is articulated to a middle degree of flexion (e.g., at about 30 degrees), the patella component 70 is moved along the patellar surface 68 and is positioned at a location 104 in the trochlear groove 66. Similarly, as the orthopaedic knee prosthesis 10 is articulated to a later degree of flexion (e.g., at about 45 degrees of flexion), the patella component 70 is positioned at a location 106 in the trochlear groove 66, as illustrated in FIG. 9. Additionally, as the orthopaedic knee prosthesis 10 is articulated to a late degree of flexion (e.g., at about 90 degrees of flexion), the patella component 70 is moved along the patellar surface 68 and is positioned in the distal region of the trochlear groove 66 at location 108, as illustrated in FIG. 12. As described in greater detail below, the trochlear groove 66 of the femoral component 12 is funnel-shaped and decreases in width between the location 102 and the location 106.

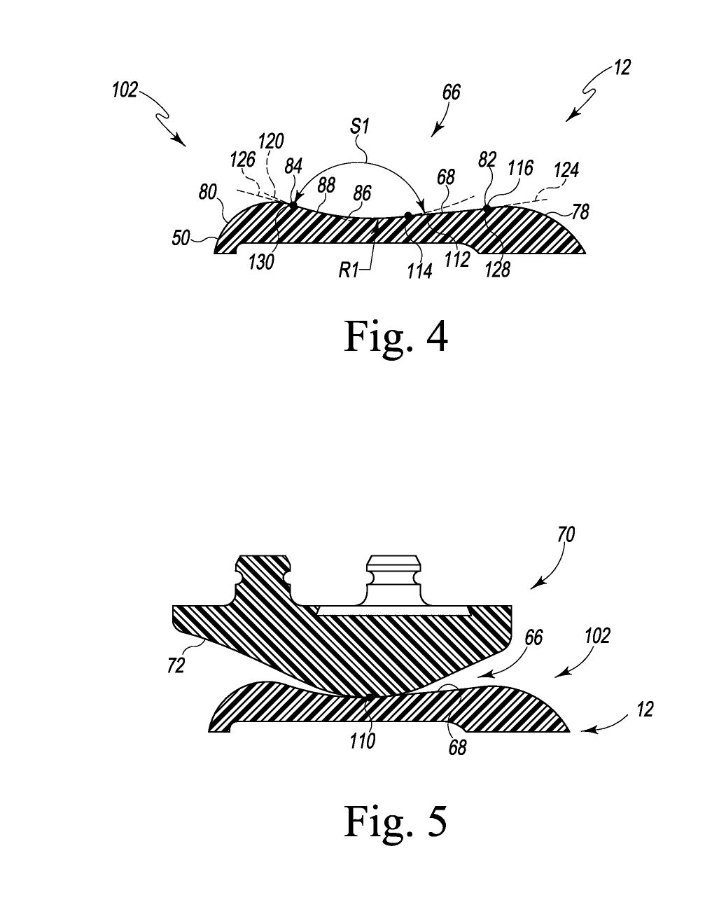

Referring now to FIGS. 3-5, the patella component 70 is positioned at a location 102 in the trochlear groove 66 when the orthopaedic knee prosthesis 10 is in extension or is otherwise not in flexion (e.g., a flexion of about 0 degrees). At the location 102, the posterior bearing surface 72 of the patella component 70 contacts the patellar surface 68 at one or more contact points 110. As shown in FIG. 4, the patellar surface 68 of the femoral component 12 at the location 102 extends between a medial anterior surface 78 and a lateral anterior surface 80 of the anterior flange 50. Each of the anterior surfaces 78, 80 is convexly curved in the coronal plane. As described above, the patellar surface 68 includes a bowed surface 88, which is concavely curved in the coronal plane and is connected to the lateral anterior surface 80 at the lateral edge 84. The patellar surface 68 also includes a medial inner surface 112 that has an end 114 connected to the bowed surface 88 and an opposite end 116 connected to the medial anterior surface 78 at the medial edge 82.

The bowed surface 88 defines an arced imaginary line 120, and the surface 88 and the line 120 define the central section 86 of the trochlear groove 66. The bowed surface 88 (and hence the arced imaginary line 120 and the central section 86) has a radius of curvature R1 equal to approximately 27 millimeters at the location 102. It should be appreciated that in other embodiments the radius of curvature may be greater than or less than 27 millimeters depending on, for example, the sizes of the femoral component 12 and the patella component 70.

As shown in FIG. 4, the trochlear groove 66 has a sulcus angle S1 that is defined between a pair of imaginary lines 124, 126. The imaginary line 124 extends along the medial inner surface 112 through a point 128 on the medial edge 82 and is tangent to the arced imaginary line 120 (and hence the bowed surface 88). The imaginary line 126 is also tangent to the arced imaginary line 120 and extends through a point 130 on the lateral edge 84. In the illustrative embodiment, the sulcus angle S1 has a magnitude of approximately 152 degrees at the location 102. It should be appreciated that in other embodiments the sulcus angle may have a different magnitude depending on, for example, the sizes of the femoral component 12 and the patella component 70.

As shown in FIG. 5, the posterior bearing surface 72 of the patella component 70 contacts the patellar surface 68 at one or more contact points 110 at the location 102. The magnitude of the angle S1 and the radius R1 result in a groove 66 that is widened and flattened relative to the bearing surface 72 of the patella component 70 such that the patella component 70 is permitted to move in the medial-lateral direction within the groove 66. As such, the patient's soft-tissues are allowed to determine the location of the patella component 70 on the patellar surface 68 at the location 102 (i.e., at a flexion of about 0 degrees).

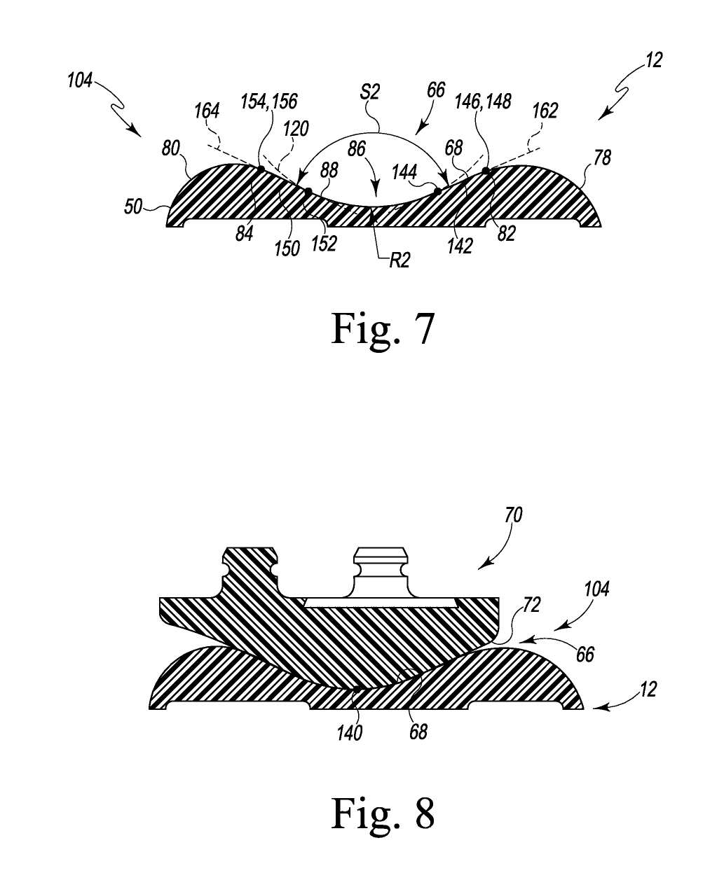

Referring now to FIGS. 6-8, the patella component 70 is positioned at a location 104 in the trochlear groove 66 at a middle degree of flexion (e.g., at about 30 degrees). At the location 104, the posterior bearing surface 72 of the patella component 70 contacts the patellar surface 68 at one or more contact points 140. As shown in FIG. 7, the patellar surface 68 of the femoral component 12 at the location 104 extends between the medial anterior surface 78 and the lateral anterior surface 80 of the anterior flange 50. Each of the anterior surfaces 78, 80 is convexly curved in the coronal plane. As described above, the patellar surface 68 includes a bowed surface 88, which is concavely curved in the coronal plane at the location 104. The patellar surface 68 also includes a medial inner surface 142 that has an end 144 connected to the bowed surface 88 and an opposite end 146 connected to the medial anterior surface 78 at a point 148 on the medial edge 82. At the location 104, the patellar surface 68 also includes a lateral inner surface 150 that has an end 152 connected to the bowed surface 88 and an opposite end 154 connected to the lateral anterior surface 80 at a point 156 on the lateral edge 84.

As described above, the bowed surface 88 defines an arced imaginary line 120, and the bowed surface 88 has a radius of curvature R1 at the location 102. At the location 104, the bowed surface 88 (and hence the arced imaginary line 120) has a radius of curvature R2 that is less than the radius R1. In other words, the central section 86 of the trochlear groove 66 has a radius of curvature at the location 104 that is less than its radius of curvature at the location 102. In the illustrative embodiment, the radius of curvature R2 is equal to approximately 15.5 millimeters. It should be appreciated that in other embodiments the radius of curvature may be greater than or less than 15.5 millimeters depending on, for example, the relative size of the femoral component and the patella component.

The trochlear groove 66 defines a sulcus angle S2 at the location 104 that is less than the sulcus angle S1 defined at the location 102. As shown in FIG. 7, the sulcus angle S2 is defined between a pair of imaginary lines 162, 164. The imaginary line 162 extends along the medial inner surface 142 through the point 148 on the medial edge 82 and is tangent to the arced imaginary line 120 (and hence the bowed surface 88). The imaginary line 164 extends along the lateral inner surface 150 through the point 156 on the lateral edge 84 and is tangent to the arced imaginary line 120. In the illustrative embodiment, the sulcus angle S2 has a magnitude of approximately 132 degrees at the location 104. It should be appreciated that in other embodiments the sulcus angle may have a different magnitude depending on, for example, the relative size of the femoral component and the patella component.

As shown in FIG. 8, the posterior bearing surface 72 of the patella component 70 contacts the patellar surface 68 at one or more contact points 140 at the location 104. Because the magnitude of the angle S2 and the radius R2 are less than the angle S1 and the radius R1, the groove 66 is more narrow and deeper at the location 104 (i.e., at a flexion of about 30 degrees) than at the location 102 (i.e., at a flexion of about 0 degrees). In that way, the groove 66 is funnel-shaped between the location 102 and the location 104. As such, the patella component 70 is more constrained and less medial-lateral movement of the patella component 70 is permitted at the location 104.

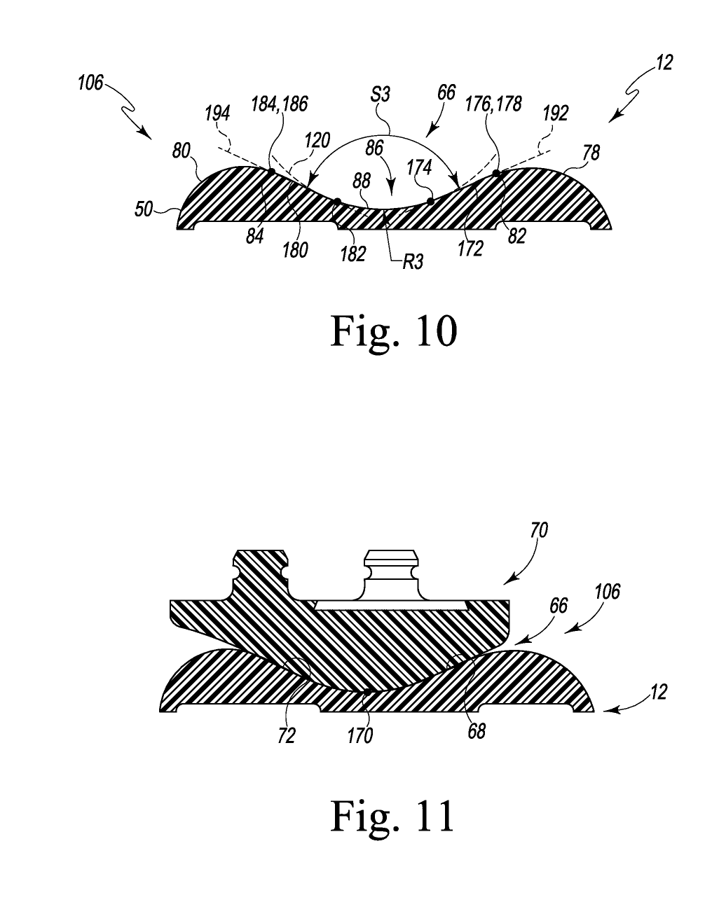

Referring now to FIGS. 9-11, the patella component 70 is positioned at a location 106 in the trochlear groove 66 at another degree of flexion (e.g., at about 45 degrees). At the location 106, the posterior bearing surface 72 of the patella component 70 contacts the patellar surface 68 at one or more contact points 170. As shown in FIG. 10, the patellar surface 68 of the femoral component 12 at the location 106 extends between the medial anterior surface 78 and the lateral anterior surface 80 of the anterior flange 50. Each of the anterior surfaces 78, 80 is convexly curved in the coronal plane. As described above, the patellar surface 68 includes a bowed surface 88, which is concavely curved in the coronal plane at the location 106. The patellar surface 68 also includes a medial inner surface 172 that has an end 174 connected to the bowed surface 88 and an opposite end 176 connected to the medial anterior surface 78 at a point 178 on the medial edge 82. At the location 106, the patellar surface 68 also includes a lateral inner surface 180 that has an end 182 connected to the bowed surface 88 and an opposite end 184 connected to the lateral anterior surface 80 at a point 186 on the lateral edge 84.

As described above, the bowed surface 88 defines an arced imaginary line 120, and the bowed surface 88 has the radii of curvature R1, R2 at the locations 102, 104, respectively. At the location 106, the bowed surface 88 (and hence the arced imaginary line 120) has a radius of curvature R3 that is less than either the radius R1 or the radius R2. Thus, the central section 86 of the trochlear groove 66 has a radius of curvature at the location 106 that is less than its radii of curvature at the locations 102, 104. In the illustrative embodiment, the radius of curvature R3 is equal to approximately 14 millimeters. It should be appreciated that in other embodiments the radius of curvature may be greater than or less than 14 millimeters depending on, for example, the relative size of the femoral component and the patella component.

The trochlear groove 66 defines a sulcus angle S3 at the location 106 that is less than either the sulcus angle S1 or the sulcus angle S2 defined at the locations 102, 104, respectively. As shown in FIG. 10, the sulcus angle S3 is defined between a pair of imaginary lines 192, 194. The imaginary line 192 extends along the medial inner surface 172 through the point 178 on the medial edge 82 and is tangent to the arced imaginary line 120 (and hence the bowed surface 88). The imaginary line 194 extends along the lateral inner surface 180 through the point 186 on the lateral edge 84 and is tangent to the arced imaginary line 120. In the illustrative embodiment, the sulcus angle S3 has a magnitude of approximately 130 degrees at the location 106. It should be appreciated that in other embodiments the sulcus angle may have a different magnitude depending on, for example, the relative size of the femoral component and the patella component.

As shown in FIG. 11, the posterior bearing surface 72 of the patella component 70 contacts the patellar surface 68 at one or more contact points 170 at the location 106. Because the magnitude of the angle S3 and the radius R3 are less than the angles S1, S2 and the radii R1, R2, the groove 66 is more narrow and deeper at the location 106 (i.e., at a flexion of about 45 degrees) than at the location 104 (i.e., at a flexion of about 30 degrees) or the location 102 (i.e., at a flexion of about 0 degrees). In that way, the groove 66 is funnel-shaped between the location 102 and the location 106. As such, the patella component 70 is more constrained and less medial-lateral movement of the patella component 70 is permitted at the location 106.

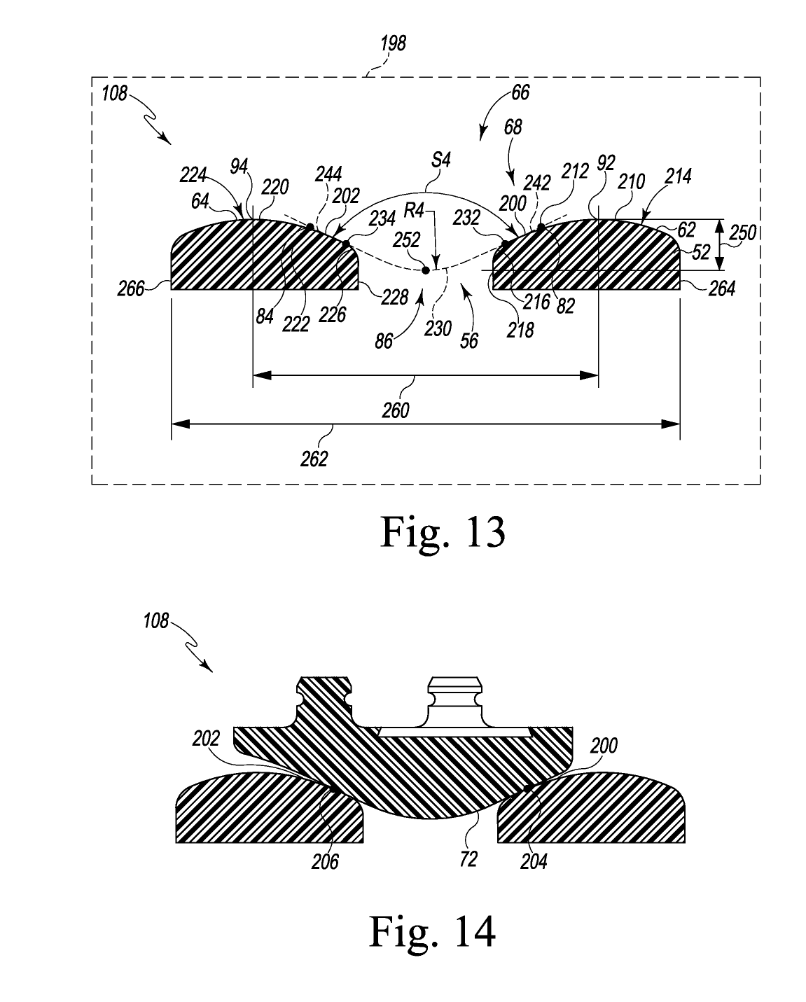

Referring now to FIGS. 12-14, the patella component 70 is positioned at a location 108 in the trochlear groove 66 at a late degree of flexion (e.g., at about 90 degrees). As shown in FIG. 13, the location 108 is positioned in a coronal plane 198 extending through the distal-most points 92, 94 of the condyles 52, 54, respectively. At the location 108, the posterior bearing surface 72 of the patella component 70 contacts the medial condyle 52 and the lateral condyle 54. The patellar surface 68 includes a medial inner surface 200 of the medial condyle 52 and a lateral inner surface 202 of the lateral condyle 54. The posterior bearing surface 72 of the patella component contacts one or more contact points 204 on the medial inner surface 200 and one or more contact points 206 on the lateral inner surface 202 at the location 108 (see FIG. 14).

As shown in FIG. 13, the medial condyle surface 62 of the medial condyle 52 has a distal-most surface 210 that is connected to the medial inner surface 200 at a point 212 on the medial edge 82. The distal-most surface 210 includes the distal-most point 92 of the medial condyle 52. The distal-most surface 210 is convexly curved in the coronal plane and has a coronal radius of curvature 214. In the illustrative embodiment, the radius of curvature 214 is equal to approximately 24.324 millimeters. It should be appreciated that in other embodiments the radius 214 may be greater than or less than 24.324 millimeters depending on the patient's bony anatomy. In the illustrative embodiment, the point 212 at which the distal-most surface 210 transitions to the medial inner surface 200 is a tangent point of the distal-most surface 210.

The medial inner surface 200 extends proximally away from the point 212. The medial inner surface 200 transitions to a rounded medial edge surface 216 that extends proximally away from the medial inner surface 200. The rounded medial edge surface 216 transitions to a flat medial surface 218 that extends proximally away from the rounded medial edge surface 216.

As shown in FIG. 13, the lateral condyle surface 64 of the lateral condyle 54 has a distal-most surface 220 that is connected to the lateral inner surface 202 at a point 222 on the lateral edge 84. The distal-most surface 220 includes the distal-most point 94 of the lateral condyle 54. The distal-most surface 220 is convexly curved in the coronal plane and has a coronal radius of curvature 224. In the illustrative embodiment, the radius of curvature 224 is equal to the radius of curvature 214 of the distal-most surface 210 of the medial condyle 52. In the illustrative embodiment, the point 222 at which the distal-most surface 220 transitions to the lateral inner surface 202 is a tangent point of the distal-most surface 220.

The lateral inner surface 202 extends proximally away from the point 222. The lateral inner surface 202 transitions to a rounded lateral edge surface 226 that extends proximally away from the lateral inner surface 202. The rounded lateral edge surface 226 transitions to a flat lateral surface 228 that extends proximally away from the rounded lateral edge surface 226. As shown in FIG. 13, the intercondylar notch 56 is defined between the flat lateral surface 228 and the flat medial surface 218.

An arced imaginary line 230 extends between the medial inner surface 200 and the lateral inner surface 202 and defines the central section 86 of the trochlear groove 66 at the location 108. The arced imaginary line 230 defines a tangent point 232 at the transition of the medial inner surface 200 and the rounded medial edge surface 216. Similarly, the arced imaginary line 230 defines another tangent point 234 at the transition of the lateral inner surface 202 and the rounded lateral edge surface 226.

As described above, the central section 86 of the trochlear groove 66 has a radius of curvature R3 at the location 106 (i.e., a flexion of about 45 degrees). At the location 108 (i.e., a flexion of about 90 degrees), the arced imaginary line 230 (and hence the central section 86) has a radius of curvature R4 that is equal to the radius of curvature R3. In the illustrative embodiment, the radius of curvature R4 is equal to approximately 14 millimeters. It should be appreciated that in other embodiments the radius of curvature may be greater than or less than 14 millimeters. It should be appreciated that in other embodiments the radius of curvature may be greater than or less than 14 millimeters depending on, for example, the relative size of the femoral component and the patella component.

The trochlear groove 66 defines a sulcus angle S4 at the location 108 that is equal to the sulcus angle S3 defined at location 106. As shown in FIG. 13, the sulcus angle S4 is defined between a pair of imaginary lines 242, 244. The imaginary line 242 is tangent to the arced imaginary line 230 and extends through the tangent point 232 and the point 212 on the medial edge 82. The imaginary line 244 is also tangent to the arced imaginary line 230 and extends through the tangent point 234 and the point 222 on the lateral edge 84. In the illustrative embodiment, the sulcus angle S4 has a magnitude of approximately 130 degrees at the location 108. It should be appreciated that in other embodiments the sulcus angle may have a different magnitude depending on, for example, the relative size of the femoral component and the patella component.

The trochlear groove 66 of the femoral component 12 has a depth 250 at the location 108 that is equal to the depth of the groove 66 at the location 106. At the location 108, the trochlear depth 250 is defined between the distal-most point 92 of the medial condyle 52 and the apex 252 of the arced imaginary line 230. In the illustrative embodiment, the depth 250 is equal to approximately 6.623 millimeters. It should be appreciated that the depth 250 may be greater than or less than the 6.623 millimeters depending on, for example, the relative size of the femoral component and the patella component.

As shown in FIG. 14, the posterior bearing surface 72 of the patella component 70 contacts the patellar surface 68 at one or more contact points 204, 206 at the location 108. Because the magnitude of the angle S4 and the radius R4 are equal to the angle S3 and the radius R3, the groove 66 is the same width and the same depth at the location 108 (i.e., at a flexion of about 90 degrees) as at the location 106 (i.e., at a flexion of about 45 degrees).

Returning to FIG. 13, the femoral component 12 has a distal coronal radial width 260 defined between the distal-most point 92 of the medial condyle 52 and the distal-most point 94 of the lateral condyle 54. In the illustrative embodiment, the radial width 260 is equal to approximately 45.398 millimeters. It should be appreciated that the radial width 260 may be greater than or less than 45.398 millimeters depending on, for example, the relative size of the femoral component and the patella component. The femoral component 12 has a component width 262 defined between the outer side surface 264 of the medial condyle 52 and the outer side surface 266 of the lateral condyle 54. In the illustrative embodiment, the component width 262 is equal to approximately 66.5 millimeters. It should be appreciated that the component width 262 may be greater than or less than 66.5 millimeters.

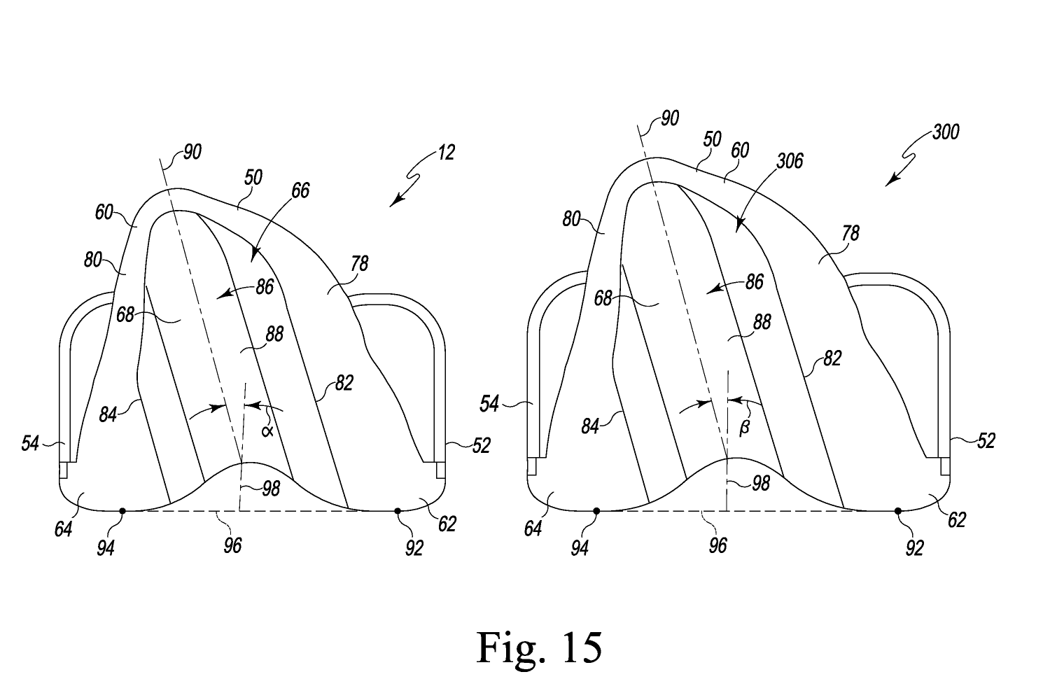

Referring now to FIGS. 15-18, a knee prosthesis assembly is typically made commercially available in a variety of different sizes, including, for example, a variety of different component widths and trochlear groove depths, to accommodate variations in patient size and anatomy across a population. For example, as shown in FIG. 15, the knee prosthesis assembly 10 may include the femoral component 12 and another femoral component 300 that is larger than the femoral component 12. While the components 12, 300 are different sizes, the component 300 has the same basic configuration as the femoral component 12. As such, some of features of the component 300 are substantially similar to those described above in reference to the femoral component 12 and are designated with the same reference numbers as those used in reference to the femoral component 12.

As shown in FIG. 15, the anterior flange 50 of the femoral component 300 includes a medial anterior surface 78 and a lateral anterior surface 80. The medial anterior surface 78 transitions to the medial condyle surface 62 of the medial condyle 52, and the lateral anterior surface 80 of the lateral condyle 54 transitions to the lateral condyle surface 64 of the lateral condyle 54. The femoral component 300 includes a patellar surface 68 that has a medial edge 82 that is connected to the medial anterior surface 78 and the medial condyle surface 62. The patellar surface 68 also has a lateral edge 84 that is connected to the lateral anterior surface 80 and the lateral condyle surface 64.

The femoral component 300 has a trochlear groove 306 that is defined by the patellar surface 68 between the edges 82, 84 thereof. The trochlear groove 306 also includes a central section 86 defined by a bowed surface 88 of the patellar surface 68. As described in greater detail below, the trochlear groove 306 has a laterally angled longitudinal axis 90 that extends through the central section 86.

The medial condyle 52 has a distal-most point 92 on the medial condyle surface 62. Similarly, the lateral condyle 54 has a distal-most point 94 on the lateral condyle surface 64. As shown in FIG. 15, the distal-most points 92, 94 are positioned in a distal transverse plane 96, and an imaginary line 98 extends orthogonal to the plane 96.

A trochlear angle β is defined between the longitudinal axis 90 of the trochlear groove 306 and the imaginary line 98. In the illustrative embodiment, the trochlear angle β has a magnitude of approximately 11.6 degrees. As described above, the femoral component 12 has a trochlear angle α that has magnitude of approximately 12.0 degrees. As such, the trochlear groove 306 of the larger component 300 is angled less than the trochlear groove 66 of the smaller component 12. It should be appreciated that in other embodiments the trochlear angle may have a magnitude in the range of 10.1 degrees to 14.1 degrees depending on, for example, the size of the femoral component.

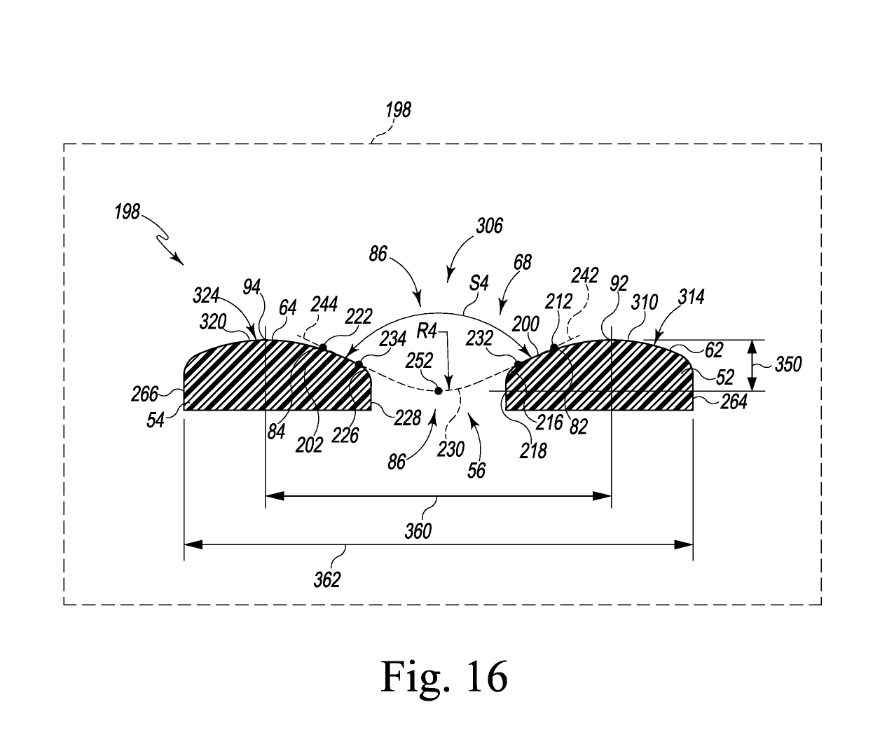

Referring now to FIG. 16, a coronal plane 198 extends through the distal-most points 92, 94 of the condyles 52, 54, respectively, of the femoral component 300. The patellar surface 68 of the femoral component 300 includes a medial inner surface 200 of the medial condyle 52 and a lateral inner surface 202 of the lateral condyle 54. The posterior bearing surface 72 of the patella component is configured to contact one or more contact points (not shown) on the medial inner surface 200 and the lateral inner surface 202.

The medial condyle surface 62 of the medial condyle 52 has a distal-most surface 310 that is connected to the medial inner surface 200 at a point 212 on the medial edge 82. As shown in FIG. 16, the distal-most surface 310 includes the distal-most point 92 of the medial condyle 52 of the femoral component 300. The distal-most surface 310 is convexly curved in the coronal plane and has a coronal radius of curvature 314. In the illustrative embodiment, the point 212 at which the distal-most surface 310 transitions to the medial inner surface 200 is a tangent point of the distal-most surface 310.

The medial inner surface 200 extends proximally away from the point 212. The medial inner surface 200 transitions to a rounded medial edge surface 216 that extends proximally away from the medial inner surface 200. The rounded medial edge surface 216 transitions to a flat medial surface 218 that extends proximally away from the rounded medial edge surface 216.

As shown in FIG. 16, the lateral condyle surface 64 of the lateral condyle 54 of the femoral component 300 has a distal-most surface 320 that is connected to the lateral inner surface 202 at a point 222 on the lateral edge 84. The distal-most surface 320 includes the distal-most point 94 of the lateral condyle 54. The distal-most surface 320 is convexly curved in the coronal plane and has a coronal radius of curvature 324. In the illustrative embodiment, the radius of curvature 324 is equal to the radius of curvature 314 of the distal-most surface 310 of the medial condyle 52. In the illustrative embodiment, the point 222 at which the distal-most surface 320 transitions to the lateral inner surface 202 is a tangent point of the distal-most surface 320.

The lateral inner surface 202 extends proximally away from the point 222. The lateral inner surface 202 transitions to a rounded lateral edge surface 226 that extends proximally away from the lateral inner surface 202. The rounded lateral edge surface 226 transitions to a flat lateral surface 228 that extends proximally away from the rounded lateral edge surface 226. As shown in FIG. 16, the intercondylar notch 56 of the femoral component 300 is defined between the flat lateral surface 228 and the flat medial surface 218.

An arced imaginary line 230 extends between the medial inner surface 200 and the lateral inner surface 202 and defines the central section 86 of the trochlear groove 66. The arced imaginary line 230 defines a tangent point 232 at the transition of the medial inner surface 200 and the rounded medial edge surface 216. Similarly, the arced imaginary line 230 defines another tangent point 234 at the transition of the lateral inner surface 202 and the rounded lateral edge surface 226.

The arced imaginary line 230 (and hence the central section 86) of the femoral component 300 has radius of curvature R4. In the illustrative embodiment, the radius of curvature R4 is equal to approximately 14 millimeters. In other words, the radius of curvature R4 of the femoral component 300 is equal to the radius of curvature R4 of the smaller femoral component 12.

Additionally, the trochlear groove 66 defines a sulcus angle S4. As shown in FIG. 16, the sulcus angle S4 is defined between a pair of imaginary lines 242, 244. The imaginary line 242 is tangent to the arced imaginary line 230 and extends through the tangent point 232 and the point 212 on the medial edge 82. The imaginary line 244 is also tangent to the arced imaginary line 230 and extends through the tangent point 234 and the point 222 on the lateral edge 84. In the illustrative embodiment, the sulcus angle S4 has a magnitude of approximately 14 degrees. In other words, the sulcus angle S4 of the femoral component 300 is equal in magnitude to the sulcus angle S4 of the femoral component 12.

As shown in FIG. 16, the trochlear groove 66 of the femoral component 300 has a depth 350, which is defined between the distal-most point 92 of the medial condyle 52 and the apex 252 of the arced imaginary line 230. The femoral component 300 also has a radial width 360 defined between the distal-most point 92 of the medial condyle 52 and the distal-most point 94 of the lateral condyle 54. The femoral component 300 has a component width 362 defined between the outer side surface 264 of the medial condyle 52 and the outer side surface 266 of the lateral condyle 54.

As described above, the trochlear depth 350 of the femoral component 300 is greater than the trochlear depth 250 of the femoral component 12. Similarly, the coronal radius of curvature 314 of the distal-most surface 310 of the medial condyle 52 of the femoral component 300 is greater than the coronal radius 214 of the femoral component 12. In the illustrative embodiment, the radius 314 of the femoral component 300 is proportionally greater than the radius 214 of the femoral component 12 by a scale factor M that is equal to 1.041. As such, the radius 314 of the femoral component 300 is equal to approximately 25.321 millimeters.

Additionally, the widths 360, 362 of the femoral component 300 are greater than the widths 260, 262 of the femoral component 12. In the illustrative embodiment, the radial width 360 of the femoral component 300 is proportionally greater than the radial width 260 of the femoral component 12 by a scale factor N of 1.024. As such, the radial width 360 of the femoral component 300 is equal to approximately 49.398 millimeters. In the illustrative embodiment, the component width 362 of the femoral component 300 is proportionally greater than the component width 262 of the femoral component 12 by a scale factor O that is equal to 1.047. As such, the component width 362 of the femoral component 300 is equal to approximately 69.626 millimeters.

While the trochlear depth 350, widths 360, 362, and coronal radius 314 of the femoral component 300 are greater than the corresponding trochlear depth 250, widths 260, 262, and coronal radius 214 of the femoral component 12, the sulcus angle S4 of the femoral component 300 is equal in magnitude to the sulcus angle S4 of the femoral component 12. Additionally, the radii R4 of the central sections 86 of the trochlear grooves 66 of the components 12, 300 are also equal. As a result, the basic configuration of the patellar surfaces 68 of the femoral components 12, 300 remains the same, thereby permitting the use of the same patella component 70 with each of the femoral components 12, 300.

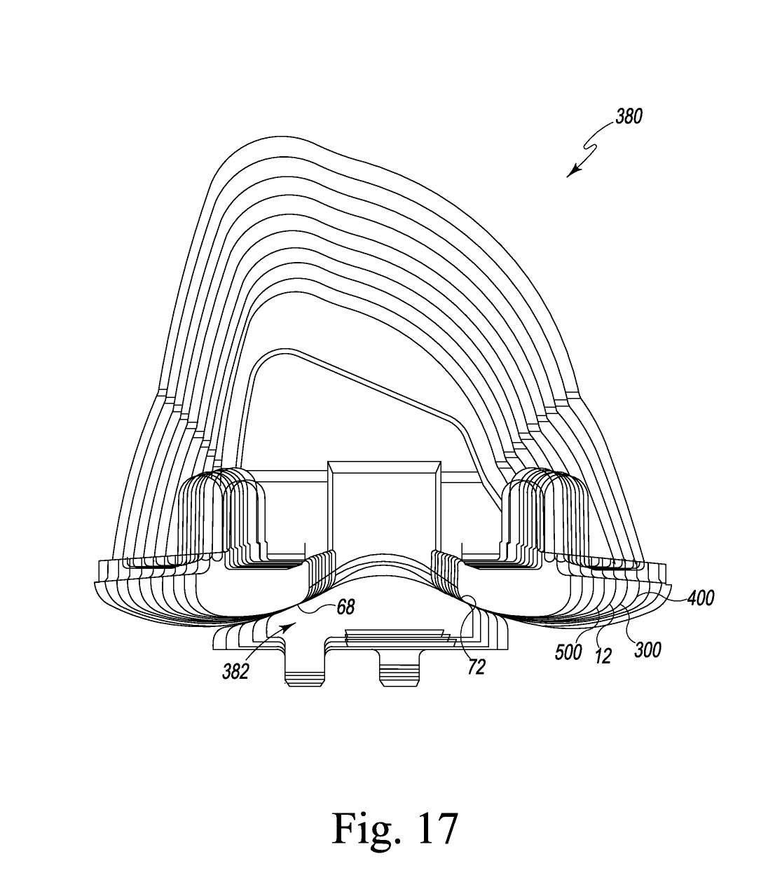

As shown in FIG. 17, the femoral components 12, 300 are shown in a diagrammatic representation with a family of differently-sized femoral components 380 and patella components 382 superimposed upon one another. As illustrated, while each of the individual femoral components 380 has a size (e.g., width, depth, or coronal radius) that is different from the other femoral components 380 of the group, the basic configuration of the patellar surfaces 68 of the femoral components 400 remains the same such that they articulate with the posterior bearing surfaces 72 of the patella components 382 across the range of differently-sized femoral components 380 and patella components 382.

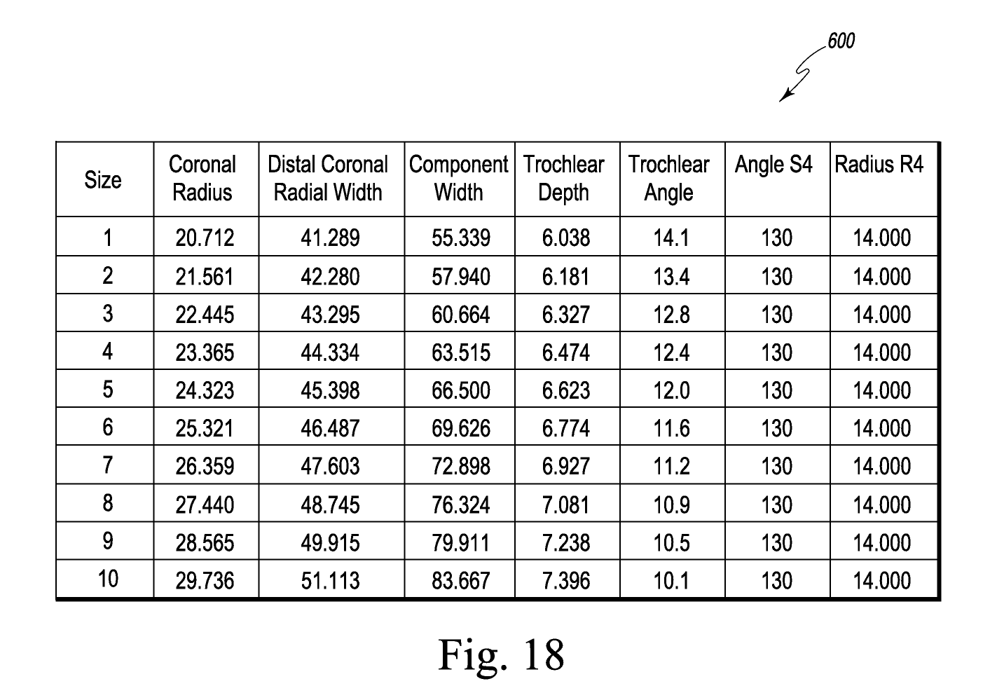

Referring now to FIG. 18, a table 600 includes the values for dimensions of the family of femoral component sizes of 1 through 10. As illustrated in the table 600, the coronal radii, distal coronal radial width, and the component width increase proportionally with each increase in component size. For example, as the femoral components increase in size, the coronal radii of the condyles 52, 54 proportionally increase.

As described above, the coronal radius 314 of the femoral component 300 is proportionally greater than the coronal radius 214 of the femoral component 12 by a scale factor M. In the table 600, the component 12 is illustratively identified as Size 5, and the component 300 is illustratively identified as Size 6. As shown in FIGS. 17 and 18, the coronal radius of the next-larger femoral component 400 (i.e., Size 7) is proportionally greater than the coronal radius 314 of the femoral component 300 by the same scale factor M, while the coronal radius of the next-smaller size femoral component 500 (i.e., Size 4) is proportionally less than the coronal radius 214 of the femoral component 12 by the same scale factor M. In the illustrative embodiment, the scale factor M is equal to 1.041. It should be appreciated that in other embodiments the scale factor M may be greater or less than 1.041 depending on the number of femoral component sizes in the component family, the variability in the size of the patients in the population, and so forth.

Additionally, the widths of the femoral components also proportionally change with each change in size. As described above, the radial width 360 of the femoral component 300 is proportionally greater than the radial width 260 of the femoral component 12 by a scale factor N. Similarly, the radial width of the next-larger femoral component 400 (i.e., Size 7) is proportionally greater than the radial width of the femoral component 300 (i.e., Size 6) by the same scale factor N, while the radial width of the next-smaller size femoral component 500 (i.e., Size 4) is proportionally less than the radial width 260 of the femoral component 12 (i.e., Size 5) by the scale factor N. In the illustrative embodiment, the scale factor N is equal to 1.024. It should be appreciated that in other embodiments the scale factor N may be greater or less than 1.024 depending on the number of femoral component sizes in the component family, the variability in the size of the patients in the population, and so forth.

As described above, the component width 362 of the femoral component 300 is proportionally greater than the component width 262 of the femoral component 12 by a scale factor O. Similarly, the component width of the next-larger femoral component 400 is proportionally greater than the component width 362 of the femoral component 300 by the same scale factor O. The component width of the next-smaller size femoral component 500 is proportionally less than the component width 262 of the femoral component 12 by the scale factor O. In the illustrative embodiment, the scale factor O is equal to 1.047. It should be appreciated that in other embodiments the scale factor O may be greater or less than 1.047 depending on the number of femoral component sizes in the family, the expected variability in the sizes of the patients in the population, and so forth.

As shown in FIG. 18, the trochlear groove depth increases with each increase in component size. The trochlear angle of the groove, however, varies inversely with each change in component size. As described above, the sulcus angle S4 and the radius R4 remain constant across the family of femoral component sizes shown in table 600, thereby permitting the femoral components 380 to articulate with the patella components 382 across the range of differently-sized femoral components 380 and patella components 382.

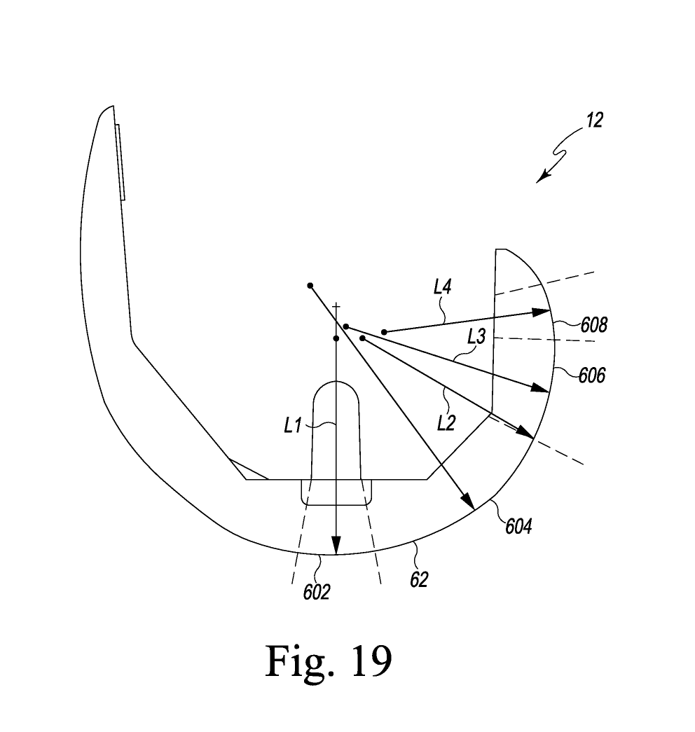

As described above, the condyles 52, 54 of the femoral component 12 include a medial condyle surface 62 and lateral condyle surface 64, respectively. In the illustrative embodiment, the condyle surfaces 62, 64 share a common sagittal geometry such that only condyle surface 62 is described in greater detail below. Referring now to FIG. 19, the condyle surface 62 is formed from a number of curved surface sections 602, 604, 606, each of which is tangent to the adjacent curved surface section. Each curved surface sections 602, 604, 606 contacts the tibial bearing 14 through different ranges of degrees of flexion. For example, the curved surface sections 602, 604 of the condyle surface 62 contact the tibial bearing 14 during early flexion. That is, as the femoral component 12 is articulated through the early degrees of flexion relative to the tibial bearing 14, the femoral component 12 contacts the tibial bearing 14 at one or more contact points on the curved surface section 602 or the curved surface section 604 at each degree of early flexion. For example, as illustrated in FIG. 3, when the femoral component 12 is positioned at about 0 degrees of flexion, the femoral component 12 contacts the bearing surface 42 of the tibial bearing 14 at a contact point 612 on the condyle surface 62.

Similarly, the curved surface section 604 of the condyle surface 62 contacts the tibial bearing 14 during mid flexion, and the curved surface section 606 of the condyle surface 600 contacts the tibial bearing 14 during late flexion. As the femoral component 12 is articulated through the middle degrees of flexion relative to the tibial bearing 14, the femoral component 12 contacts the tibial bearing 14 at one or more contact points on the curved surface section 604 at each degree of mid flexion. For example, as illustrated in FIG. 6, when the femoral component 12 is positioned at about 30 degrees of flexion, the femoral component 12 contacts the bearing surface 42 of the tibial bearing 14 at a contact point 614 on the condyle surface 62. Additionally, as the femoral component 12 is articulated through the late degrees of flexion relative to the tibial bearing 14, the femoral component 12 contacts the tibial bearing 14 at one or more contact points on the curved surface section 606 at each degree of late flexion. For example, as illustrated in FIG. 12, when the femoral component 12 is positioned at about 90 degrees of flexion, the femoral component 12 contacts the bearing surface 42 of the tibial bearing 14 at a contact point 616 on the condyle surface 62. Of course, it should be appreciated that the femoral component 12 contacts the tibial bearing 14 at a plurality of contact points on the condyle surface 62 at any one particular degree of flexion. However, for clarity of description, only the contact points 612, 614, 616, have been illustrated in FIGS. 3, 6, and 12, respectively.

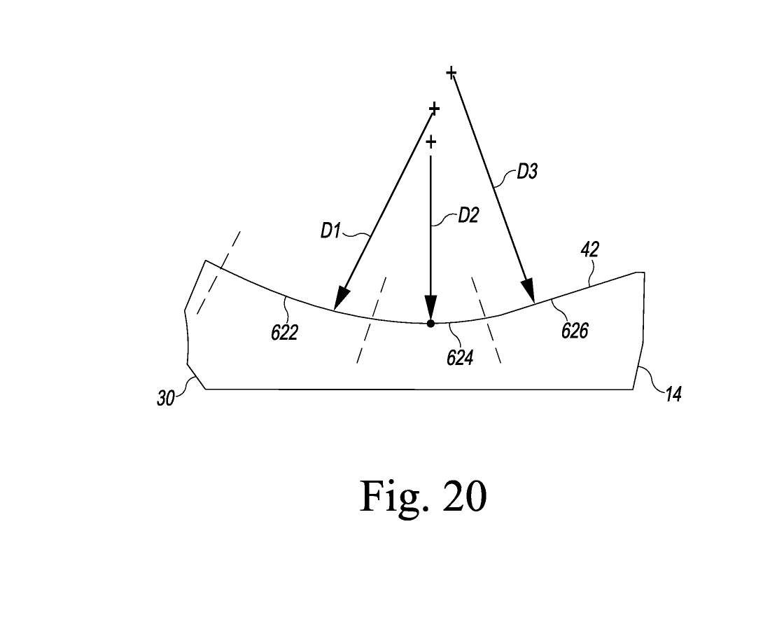

As described above, the tibial bearing 14 includes a medial bearing surface 42 and a lateral bearing surface 44 configured to engage the condyle surfaces 62, 64, respectively, of the femoral component 12. In the illustrative embodiment, the bearing surfaces 42, 44 share a common sagittal geometry such that only bearing surface 42 is described in greater detail below. Referring now to FIG. 20, the tibial bearing 14 the bearing surface 42 is formed from a number of curved surface sections 622, 624, 626, each of which is tangent to the adjacent curved surface section. Each of the curved surface sections 622, 624, 626 of the bearing surface 42 is defined by a constant radius of curvature D1, D2, D3, respectively. As described in greater detail below, each curved surface sections 602, 604, 606, and 608 of the femoral component 12 contacts different curved surface sections 622, 624, 626 of the tibial bearing 14 through different ranges of degrees of flexion.

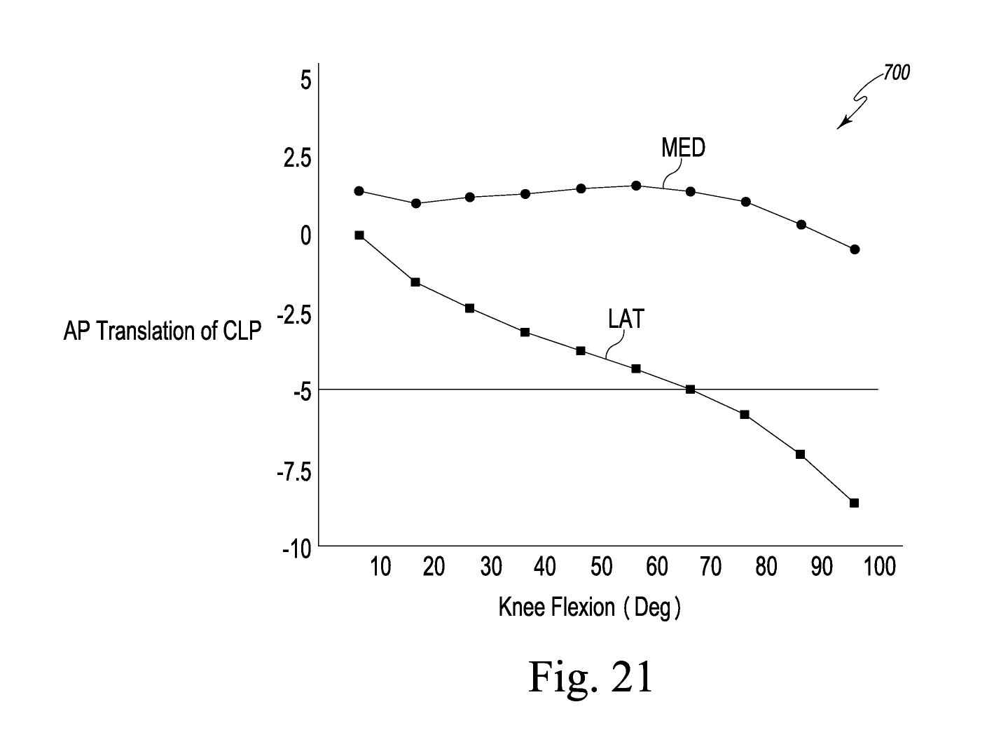

Referring now to FIG. 21, a graph 700 shows the anterior-posterior translation of the condylar lowest or most distal points (CLP) of the medial condyle 52 (“med”) and the lateral condyle 54 (“lat”) during deep knee bending In graph 700, a downwardly sloped line represents posterior roll-back of the femoral component 12 on the tibial bearing 14 and an upwardly sloped line represents anterior translation of the femoral component 12 on the tibial bearing 14.

As shown in graph 700, the lateral condyle 52 of femoral component 12 gradually rolls back posteriorly on the tibial bearing 14 as the orthopaedic prosthesis 10 is moved through the range of flexion. The medial condyle 54 also rolls back in early flexion but then moves anteriorly during mid flexion. The medial condyle 54 then continues rolling back posteriorly during later flexion.

When the medial condyle 54 or lateral condyle 52 moves anteriorly, different sections of the femoral component 12 may contact the curved surface section 624 of the tibial bearing 14. As the femoral component 12 rolls back on the tibial bearing 14, different sections of the femoral component 12 may contact the section 624 or the section 626 of the tibial bearing 14. For example, in one embodiment, the curved surface section 602 of the condyle surface 62 may contact the curved surface section 624 of the tibial bearing 14 during early flexion. That is, as the femoral component 12 is articulated through the early degrees of flexion (i.e., less than 30 degrees) relative to the tibial bearing 14, the femoral component 12 may contact the curved surface section 624 of the tibial bearing 14 at one or more contact points 612 at each degree of early flexion. The radii L1, D2 of curvature of the component 12 and bearing 14 may define a ratio of L1/D2 that corresponds to the sagittal conformity of the femoral component 12 and the bearing 14 at the contact point 612. In one embodiment, the ratio of L1/D2 is approximately 0.88.

Beyond early flexion, the curved surface sections 604, 606, 608 of the condyle surface 62 may contact the curved surface section 626 of the tibial bearing 14. The radii L2, D3 of the sections 604, 626 of the component 12 and the bearing 14 may define a ratio of L2/D3 that corresponds to the sagittal conformity of the femoral component 12 and the bearing 14 at the contact point 614 (i.e., at 30 degrees of flexion). In one embodiment, the ratio of L2/D3 is approximately 0.46. The radii L3, D3 of the sections 604, 626 of the component 12 and the bearing 14 may define a ratio of L3/D3 that corresponds to the sagittal conformity of the femoral component 12 and the bearing 14 at the contact point 616 (i.e., at 90 degrees of flexion). In one embodiment, the ratio of L2/D3 is approximately 0.40.

Depending on the kinematics of the patient's knee, the femoral component 12 may roll back more gradually such that the curved surface sections 602, 604 of the condyle surface 62 may contact the curved surface section 624 of the tibial bearing 14 during early flexion to mid flexion (i.e., less than 60 degrees). Beyond mid flexion, the curved surface sections 606, 608 of the condyle surface 62 may contact the curved surface section 626 of the tibial bearing 14.

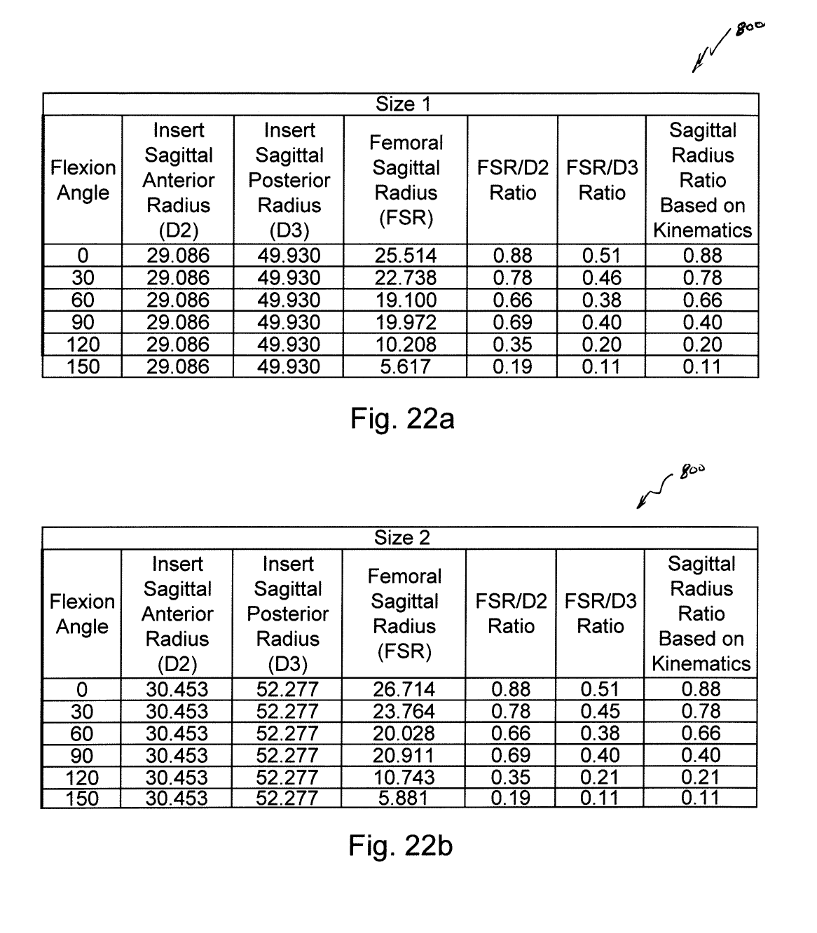

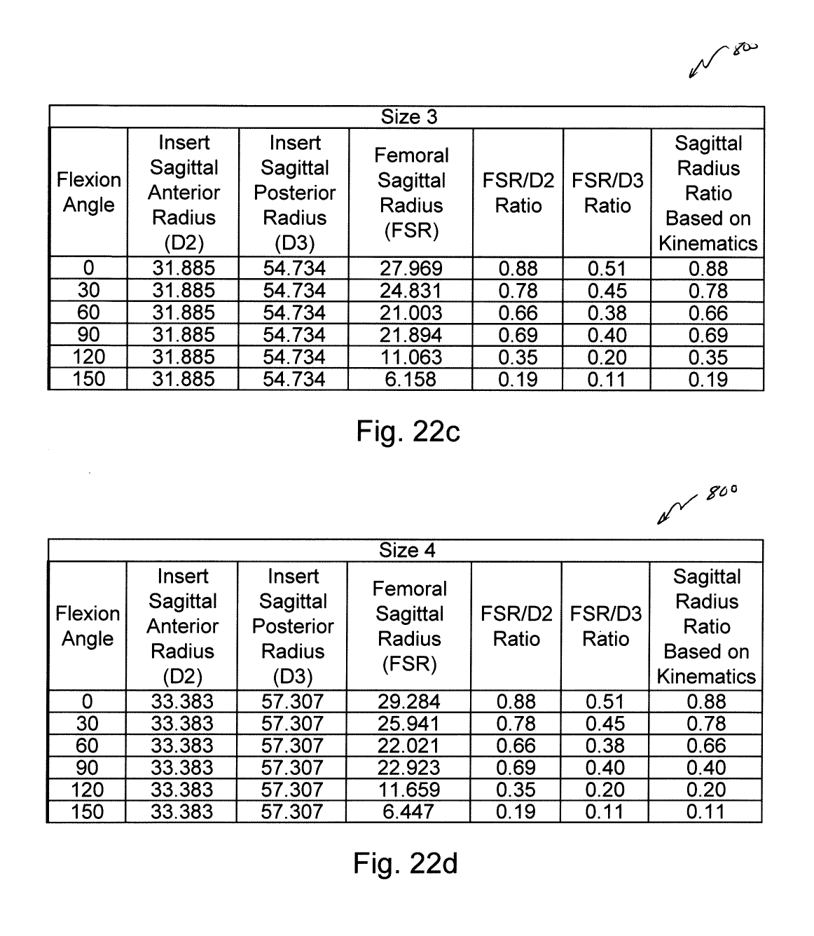

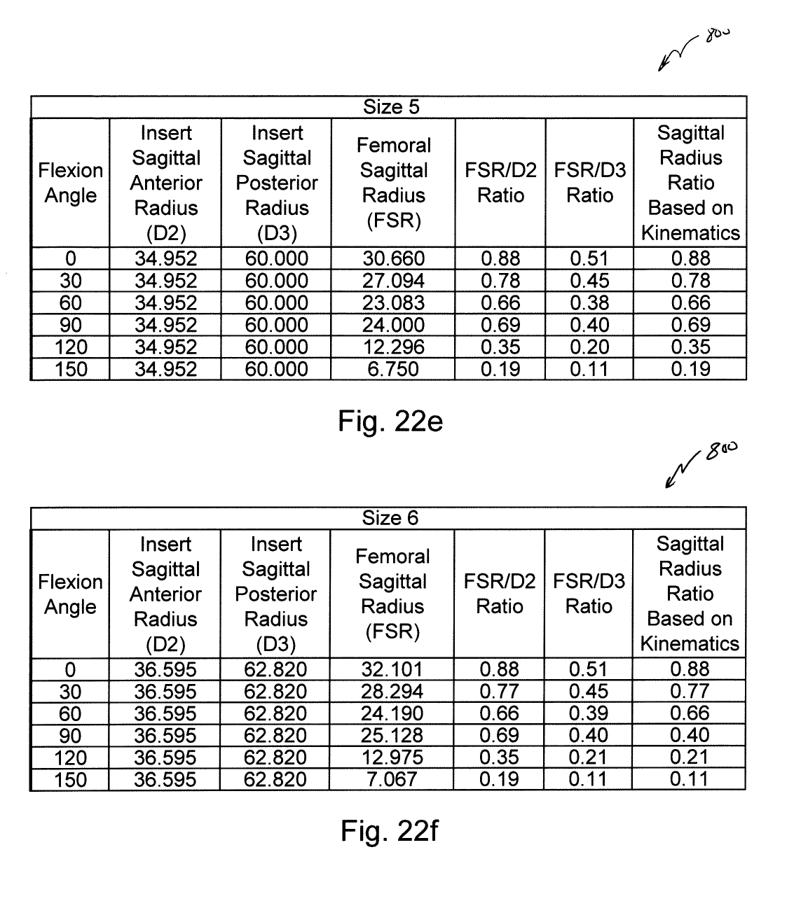

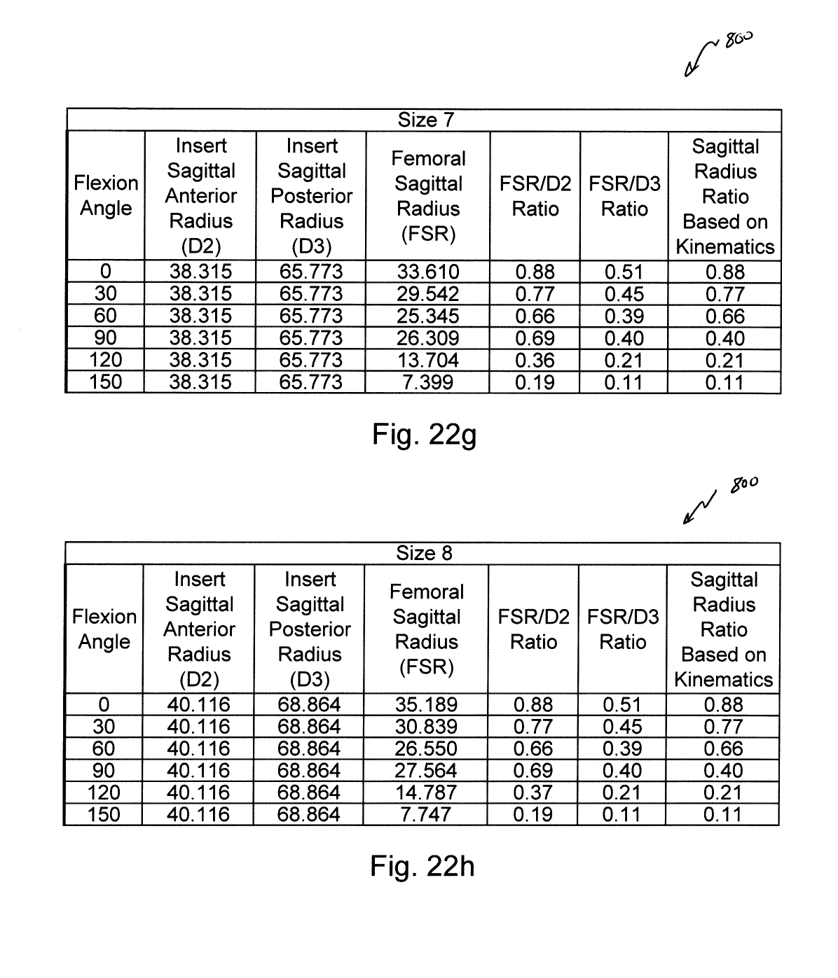

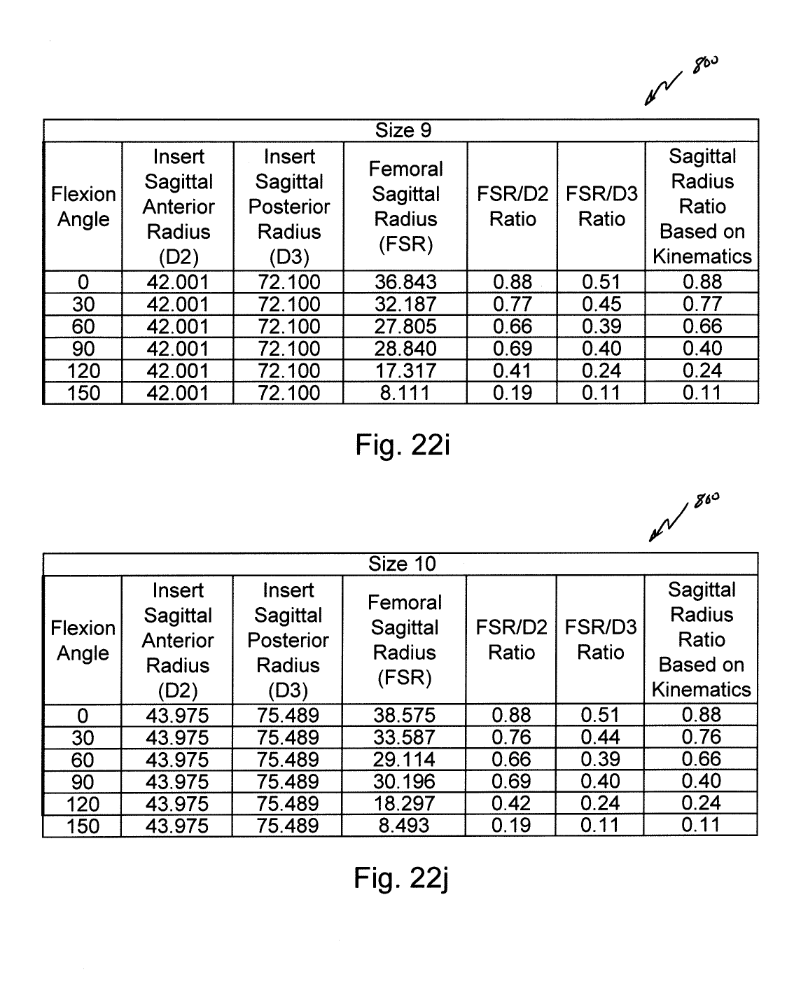

Referring now to FIG. 22, a table 800 defines the length of each sagittal radii of curvature of the femoral component 12 and the length of each sagittal radii of curvature of the tibial bearing 14 for a family of femoral component and tibial bearing sizes. In the illustrative embodiment, the femoral component 12 is a cruciate retaining femoral component. As shown in table 800, the sagittal conformity decreases as the orthopaedic prosthesis 10 moves from 0 degrees of flexion through 60 degrees of flexion, regardless of the effect of the kinematics. In later flexion (i.e., around 90 degrees), the conformity increases slightly before decreasing in late flexion. The sagittal conformity is greater when the femoral component 12 engages the curved surface section 624 (i.e., the anterior radius) of the tibial bearing 14. However, the ratios of the sagittal radii are constant across the various sizes of components and tibial bearings such that the sagittal conformities at 0 degrees of flexion for a size 1 implant are the same as the sagittal conformities at 0 degrees flexion for a size 2 implant.

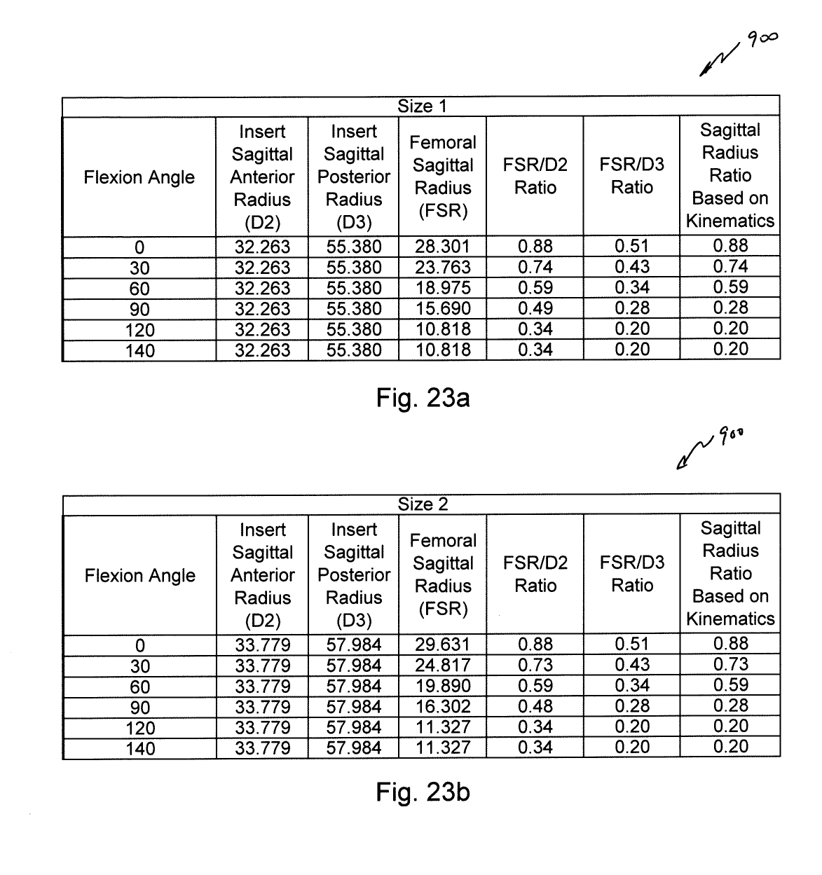

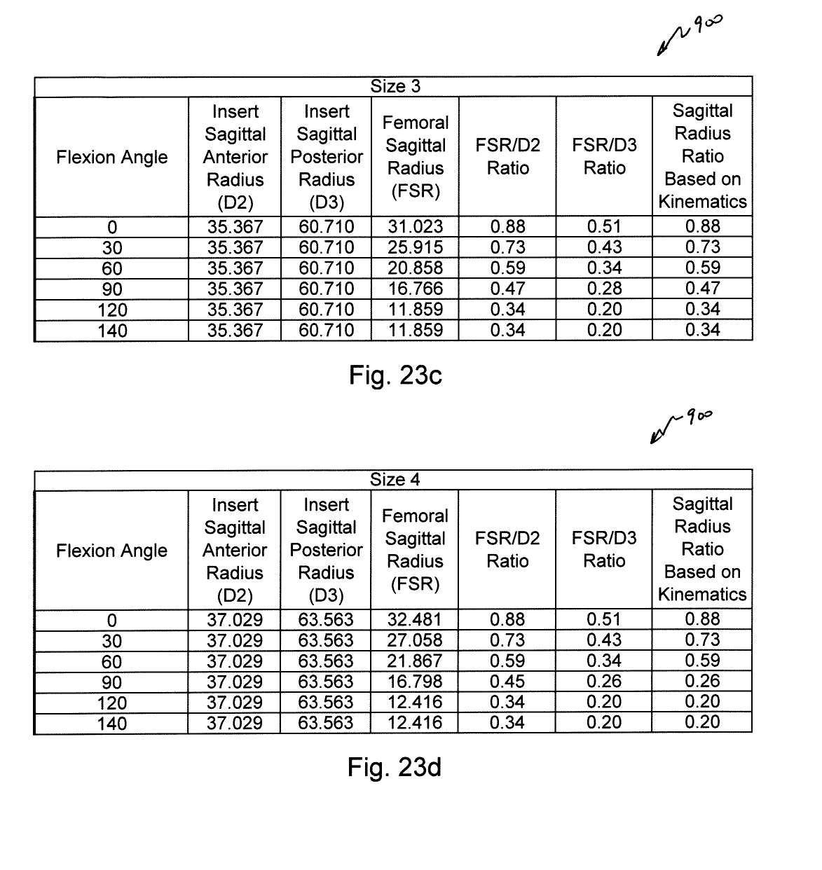

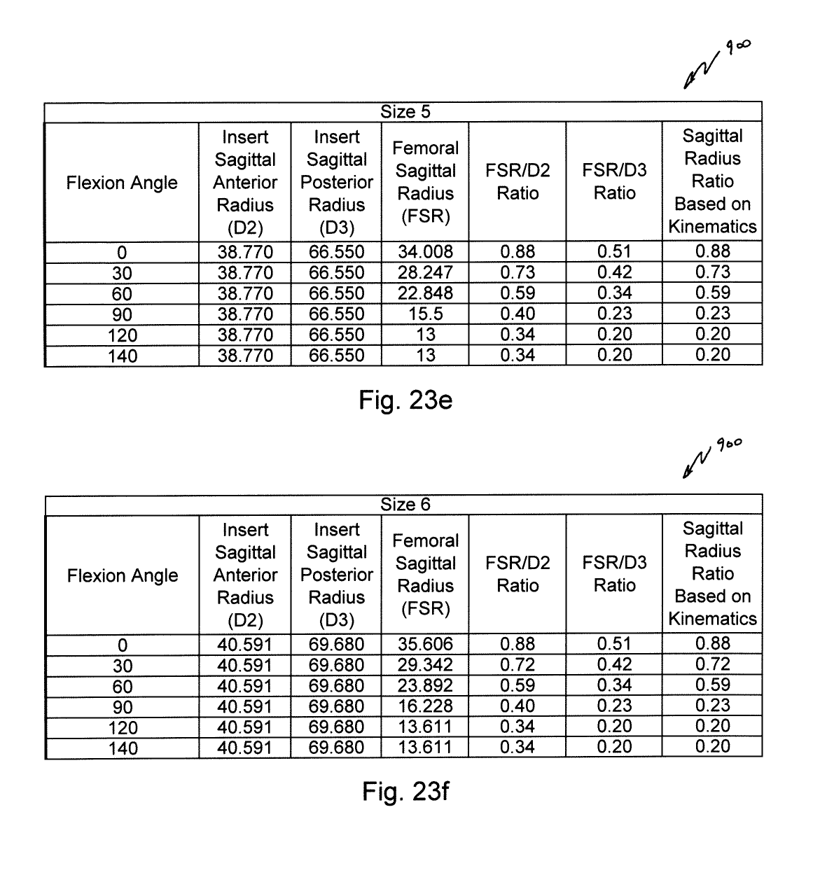

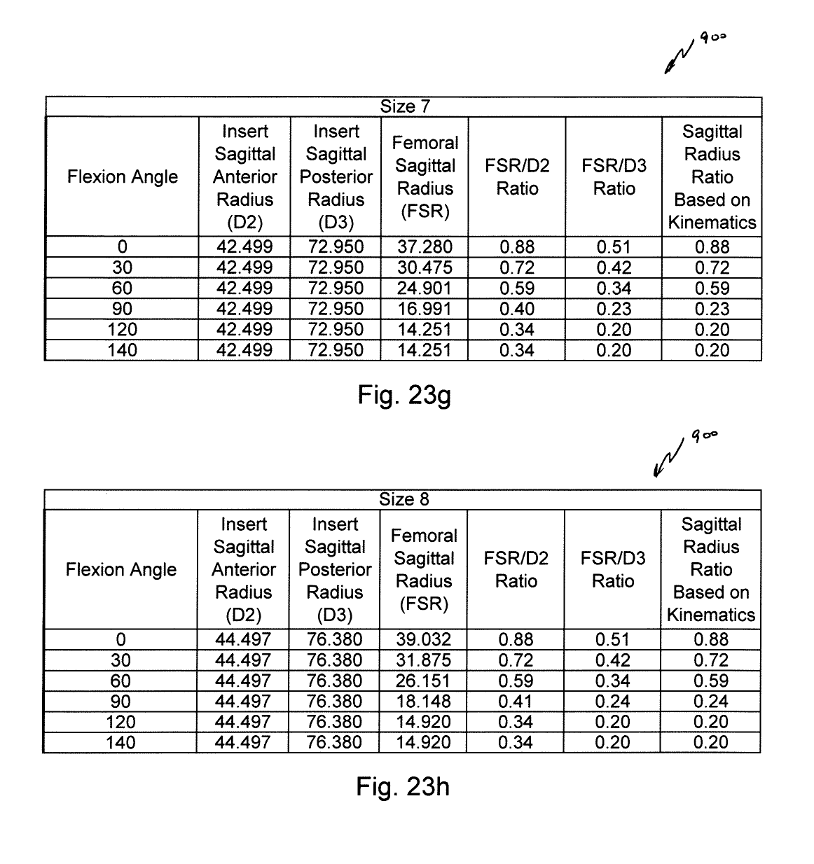

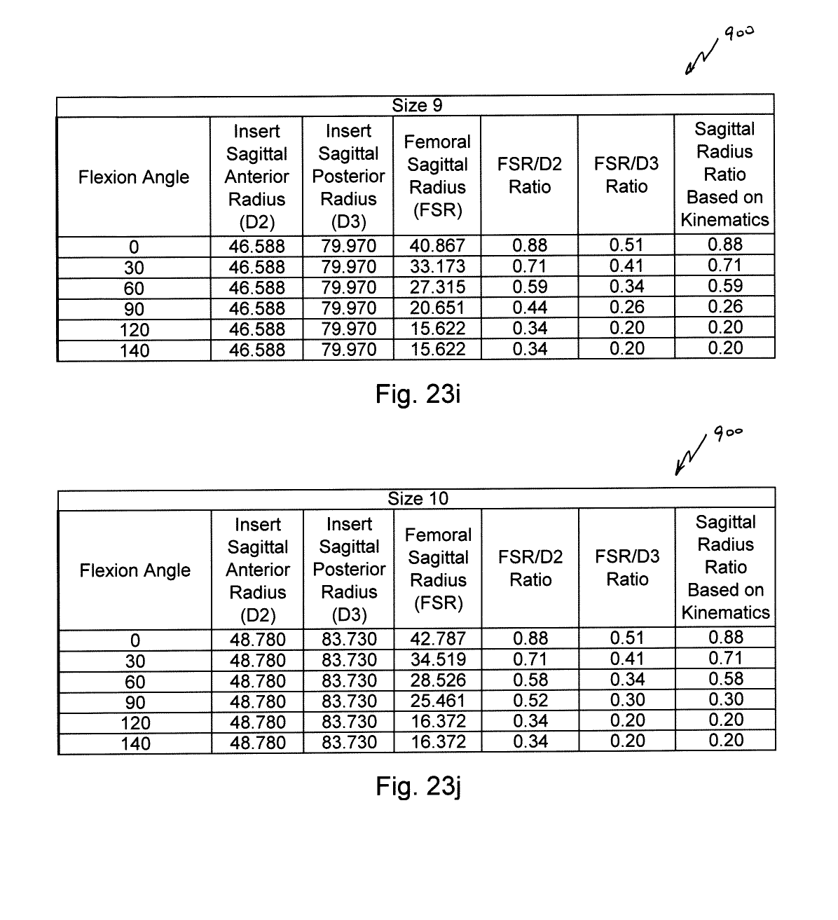

Referring now to FIG. 23, a table 900 defines the length of each sagittal radii of curvature of the femoral component 12 and the length of each sagittal radii of curvature of the tibial bearing 14 for another family of femoral component and tibial bearing sizes. In the illustrative embodiment, the femoral component 12 is a posterior-stabilized femoral component. As shown in table 900, the sagittal conformity decreases as the orthopaedic prosthesis 10 moves from 0 degrees of flexion through 60 degrees of flexion, regardless of the effect of the kinematics. The sagittal conformity is greater when the femoral component 12 engages the curved surface section 624 (i.e., the anterior radius) of the tibial bearing 14. However, the ratios of the sagittal radii are constant across the various sizes of components and tibial bearings such that the sagittal conformities at 0 degrees of flexion for a size 1 implant are the same as the sagittal conformities at 0 degrees flexion for a size 2 implant.

While the disclosure has been illustrated and described in detail in the drawings and foregoing description, such an illustration and description is to be considered as exemplary and not restrictive in character, it being understood that only illustrative embodiments have been illustrated and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic knee prosthesis assembly, comprising:
a plurality of femoral components, each component including a medial condyle and a lateral condyle, wherein when each component is viewed in a coronal plane extending through a distal-most point of the medial condyle and a distal-most point of the lateral condyle:
the medial condyle has (i) a medial distal-most surface that is curved and includes the distal-most point of the medial condyle, the medial distal-most surface having a coronal radius of curvature, and (ii) a medial inner surface connected to the medial distal-most surface and extending proximally away from the medial distal-most surface,
the lateral condyle has (i) a lateral distal-most surface that includes the distal-most point of the lateral condyle, and (ii) a lateral inner surface connected to the lateral distal-most surface and extending proximally away from the lateral distal-most surface,
an angle is defined between the medial inner surface and the lateral inner surface, and
wherein (i) the plurality of femoral components include a first component, a second component, and a third component, (ii) the angles of the first, second, and third components are equal in magnitude, (iii) the coronal radius of the first component is greater than the coronal radius of the second component by a scale factor, and (iv) the coronal radius of the second component is greater than the coronal radius of the third component by the scale factor.

2. The orthopaedic knee prosthesis assembly of claim 1, wherein the scale factor is equal to 1.041.

3. The orthopaedic knee prosthesis assembly of claim 1, wherein, when each component is viewed in the coronal plane, the lateral distal-most surface is curved and has a coronal radius of curvature that is equal to the coronal radius of curvature of the medial distal-most surface.

4. The orthopaedic knee prosthesis assembly of claim 1, wherein:
the scale factor is a first scale factor,
when each component is viewed in the coronal plane, a width is defined between the distal-most point of the medial condyle and the distal-most point of the lateral condyle,
the width of the first component is greater than the width of the second component by a second scale factor different from the first scale factor, and
the width of the second component is greater than the width of the third component by the second scale factor.

5. The orthopaedic knee prosthesis assembly of claim 1, wherein the second scale factor is equal to approximately 1.024.

6. The orthopaedic knee prosthesis assembly of claim 1, wherein the magnitude of each of the angles of the first, second, and third components is approximately 130 degrees.

7. The orthopaedic knee prosthesis assembly of claim 1, wherein when each component is viewed in the coronal plane:
the medial condyle has a medial rounded edge surface that is connected to the medial inner surface and extends proximally away from the medial inner surface,
the lateral condyle has a lateral rounded edge surface that is connected to the lateral inner surface and extends proximally away from the lateral inner surface, and
an arced imaginary line extends between the medial condyle and the lateral condyle and has a radius of curvature, the arced imaginary line defining (i) a first tangent point at the transition between the medial rounded edge surface and the medial inner surface and (ii) a second tangent point at the transition between the lateral rounded edge surface and the lateral inner surface,
wherein the radii of curvature of the arced imaginary lines of the first, second, and third components are equal.

8. The orthopaedic knee prosthesis assembly of claim 7, wherein the radius of curvature of the arced imaginary line of each of the first, second, and third components is equal to approximately 14 millimeters.

9. The orthopaedic knee prosthesis assembly of claim 7, wherein when each component is viewed in the coronal plane:
the medial condyle has a medial flat surface that is connected to the medial rounded edge surface and extends proximally away from the medial rounded edge surface,
the lateral condyle has a lateral flat surface that is connected to the lateral rounded edge surface and extends proximally away from the lateral rounded edge surface, and
each component includes an intercondylar notch defined between the medial flat surface and the lateral flat surface.

10. The orthopaedic knee prosthesis assembly of claim 7, wherein:
when each femoral component is viewed in the coronal plane, the arced imaginary line, the medial inner surface, and the lateral inner surface define a trochlear groove of the component, the trochlear groove having a depth,
the depth of the trochlear groove of the first component is greater than the depth of the trochlear groove of the second component, and
the depth of the trochlear groove of the second component is greater than the depth of the trochlear groove of the third component.

11. The orthopaedic knee prosthesis assembly of claim 10, wherein when each femoral component is viewed in the coronal plane:
the arced imaginary line has an apex, and
the depth of the trochlear groove is defined between the distal-most point of the medial condyle and the apex of the arced imaginary line.

12. An orthopaedic knee prosthesis assembly, comprising:
a plurality of femoral components, each component including a medial condyle and a lateral condyle, wherein when each component is viewed in a coronal plane extending through a distal-most point of the medial condyle and a distal-most point of the lateral condyle:
the medial condyle has (i) a medial distal-most surface that is curved and includes the distal-most point of the medial condyle, the medial distal-most surface having a coronal radius of curvature, and (ii) a medial inner surface extending proximally away from the medial distal-most surface,
the lateral condyle has (i) a lateral distal-most surface that includes the distal-most point of the lateral condyle, and (ii) a lateral inner surface extending proximally away from the lateral distal-most surface,
an arced imaginary line has a first tangent point on the medial inner surface and a second tangent point on the lateral inner surface,
a first imaginary line extends through the first tangent point of the arced imaginary line and a third tangent point that is defined at a transition between the medial inner surface and the medial distal-most surface,
a second imaginary line extends through the second tangent point of the arced imaginary line and a fourth tangent point that is defined at a transition between the lateral inner surface and the lateral distal-most surface, and
an angle is defined between the first imaginary line and the second imaginary line,
wherein (i) the plurality of femoral components includes a first component, a second component, and a third component, (ii) the angles of the components are equal in magnitude, (iii) the coronal radius of the first component is greater than the coronal radius of the second component by a scale factor, and (iv) the coronal radius of the second component is greater than the coronal radius of the third component by the scale factor.

13. The orthopaedic knee prosthesis assembly of claim 12, wherein the scale factor is equal to approximately 1.041.

14. The orthopaedic knee prosthesis assembly of claim 12, wherein:
when each component is viewed in the coronal plane, a width is defined between the distal-most point of the medial condyle and the distal-most point of the lateral condyle,
the width of the first component is greater than the width of the second component by a second scale factor, and
the width of the second component is greater than the width of the third component by the second scale factor.

15. The orthopaedic knee prosthesis assembly of claim 12, wherein each arced imaginary line has a radius of curvature, and the radii of curvature of the arced imaginary lines of the first, second, and third components are equal.

16. The orthopaedic knee prosthesis assembly of claim 15, wherein (i) the magnitude of each of the angles of the plurality of femoral components is approximately 130 degrees, and (ii) the radii of curvature of each of the arced imaginary lines of the plurality of femoral components is approximately 14 millimeters.

17. An orthopaedic knee prosthesis assembly, comprising:
a plurality of femoral components, each component including a medial condyle and a lateral condyle,
wherein when each component is viewed in a coronal plane extending through a distal-most point of the medial condyle and a distal-most point of the lateral condyle: (i) the medial condyle has a medial curved distal-most surface that includes the distal-most point, the medial curved distal-most surface having a coronal radius of curvature, and (ii) a width is defined between the distal-most points of the medial condyle and the lateral condyle,
wherein (i) the plurality of femoral components include a first, second, and third component, (ii) the coronal radius of the first component is greater than the coronal radius of the second component by a first scale factor, (iii) the coronal radius of the second component is greater than the coronal radius of the third component by the first scale factor, (iv) the width of the first component is greater than the width of the second component by a second scale factor that is less than the first scale factor, and (v) the width of the second component is greater than the width of the third component by the second scale factor.

18. The orthopaedic knee prosthesis assembly of claim 17, wherein the first scale factor is equal to 1.041.

19. The orthopaedic knee prosthesis assembly of claim 18, wherein the second scale factor is equal to 1.024.

20. The orthopaedic knee prosthesis assembly of claim 17, wherein when each femoral component is viewed in the coronal plane:
the medial condyle has (i) a medial inner surface extending proximally away from the medial curved distal-most surface and (ii) a medial rounded edge surface extending proximally away from the medial inner surface, and
an arced imaginary line has a first tangent point at a transition between the medial rounded edge surface and the medial inner surface, the arced imaginary line having a radius of curvature,
wherein the radii of curvature of the arced imaginary lines of the first, second, and third components are equal.
